SEQUENCE LISTING (18 sequences)

SEQ ID NO: 1 - DNA, 1997 bp, Homo sapiens
  misc_feature: VEGF-C cDNA
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 2 - protein, 419 aa, Homo sapiens
  MISC_FEATURE: VEGF-C AA
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 3 - DNA, 2029 bp, Homo sapiens
  misc_feature: VEGF-D cDNA
gttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 4 - protein, 354 aa, Homo sapiens
  MISC_FEATURE: VEGF-D AA
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 5 - DNA, 1997 bp, Homo sapiens
  misc_feature: VEGF-C delta C156 cDNA
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccannngtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 6 - protein, 419 aa, Homo sapiens
  MISC_FEATURE: VEGF-C delta C156 AA
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPXVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 7 - DNA, 1325 bp, Mus musculus
  misc_feature: VEGF-D D1 cDNA
ggagaatgccttttgcaacacttttcagtagctgcctggaaacaactgcttagtcatcggtagacatttaaaatattcaaaatgtatggagaatggggaatggggaatatcctcatgatgttccatgtgtacttggtgcagggcttcaggagcgaacatggaccagtgaaggatttttcttttgagcgatcatcccggtccatgttggaacgatctgaacaacagatccgagcagcttctagtttggaggagttgctgcaaatcgcgcactctgaggactggaagctgtggcgatgccggttgaagctcaaaagtcttgccagtatggactcacgctcagcatcccatcgctccaccagatttgcggcaactttctatgacactgaaacactaaaagttatagatgaagaatggcagaggacccaatgcagccctagagagacatgcgtagaagtcgccagtgagctggggaagacaaccaacacattcttcaagcccccctgtgtaaatgtcttccggtgtggaggctgctgcaacgaagagggtgtgatgtgtatgaacacaagcacctcctacatctccaaacagctctttgagatatcagtgcctctgacatcagtgcccgagttagtgcctgttaaaattgccaaccatacgggttgtaagtgcttgcccacgggcccccgccatccttactcaattatcagaagatccattcagaccccagaagaagatgaatgtcctcattccaagaaactctgtcctattgacatgctgtgggataacaccaaatgtaaatgtgttttgcaagacgagactccactgcctgggacagaagaccactcttacctccaggaacccactctctgtggaccgcacatgacgtttgatgaagatcgctgtgagtgcgtctgtaaagcaccatgtccgggagatctcattcagcacccggaaaactgcagttgctttgagtgcaaagaaagtctggagagctgctgccaaaagcacaagatttttcacccagacacctgcagctgtgaggacagatgtccttttcacaccagaacatgtgcaagtagaaagccagcctgtggaaagcactggcgctttccaaaggagacaagggcccagggactctacagccaggagaacccttgattcaacttcctttcaagtccccccatctctgtcattttaaacagctcactgctttgtcaagttgctgtcactgttgcccactaccccttgaacatgtgcaaacacagacacacacacacacacacacacagagcaactagaattatgttttctaggtgctgcctaag

SEQ ID NO: 8 - protein, 358 aa, Mus musculus
  MISC_FEATURE: VEGF-D D1 AA
MYGEWGMGNILMMFHVYLVQGFRSEHGPVKDFSFERSSRSMLERSEQQIRAASSLEELLQIAHSEDWKLWRCRLKLKSLASMDSRSASHRSTRFAATFYDTETLKVIDEEWQRTQCSPRETCVEVASELGKTTNTFFKPPCVNVFRCGGCCNEEGVMCMNTSTSYISKQLFEISVPLTSVPELVPVKIANHTGCKCLPTGPRHPYSIIRRSIQTPEEDECPHSKKLCPIDMLWDNTKCKCVLQDETPLPGTEDHSYLQEPTLCGPHMTFDEDRCECVCKAPCPGDLIQHPENCSCFECKESLESCCQKHKIFHPDTCSCEDRCPFHTRTCASRKPACGKHWRFPKETRAQGLYSQENP

SEQ ID NO: 9 - DNA, 1135 bp, Mus musculus
  misc_feature: VEGF-D D2 cDNA
aaactttgcttctggagaatgccttttgcaacacttttcagtagctgcctggaaacaactgcttagtcatcggtagacatttaaaatattcaaaatgtatggagaatggggaatggggaatatcctcatgatgttccatgtgtacttggtgcagggcttcaggagcgaacatggaccagtgaagcgatcatcccggtccatgttggaacgatctgaacaacagatccgagcagcttctagtttggaggagttgctgcaaatcgcgcactctgaggactggaagctgtggcgatgccggttgaagctcaaaagtcttgccagtatggactcacgctcagcatcccatcgctccaccagatttgcggcaactttctatgacactgaaacactaaaagttatagatgaagaatggcagaggacccaatgcagccctagagagacatgcgtagaagtcgccagtgagctggggaagacaaccaacacattcttcaagcccccctgtgtaaatgtcttccggtgtggaggctgctgcaacgaagagggtgtgatgtgtatgaacacaagcacctcctacatctccaaacagctctttgagatatcagtgcctctgacatcagtgcccgagttagtgcctgttaaaattgccaaccatacgggttgtaagtgcttgcccacgggcccccgccatccttactcaattatcagaagatccattcagaccccagaagaagatgaatgtcctcattccaagaaactctgtcctattgacatgctgtgggataacaccaaatgtaaatgtgttttgcaagacgagactccactgcctgggacagaagaccactcttacctccaggaacccactctctgtggaccgcacatgacgtttgatgaagatcgctgtgagtgcgtctgtaaagcaccatgtccgggagatctcattcagcacccggaaaactgcagttgctttgagtgcaaagaaagtctggagagctgctgccaaaagcacaagatttttcacccagacacctgcaggtcaatggtcttttcgctttccccttaacttggtttactgatgacatttaaaggacatactaatctgatctgttcaggctcttttctctcagagtccaagcac

SEQ ID NO: 10 - protein, 321 aa, Mus musculus
  MISC_FEATURE: VEGF-D D2 AA
MYGEWGMGNILMMFHVYLVQGFRSEHGPVKRSSRSMLERSEQQIRAASSLEELLQIAHSEDWKLWRCRLKLKSLASMDSRSASHRSTRFAATFYDTETLKVIDEEWQRTQCSPRETCVEVASELGKTTNTFFKPPCVNVFRCGGCCNEEGVMCMNTSTSYISKQLFEISVPLTSVPELVPVKIANHTGCKCLPTGPRHPYSIIRRSIQTPEEDECPHSKKLCPIDMLWDNTKCKCVLQDETPLPGTEDHSYLQEPTLCGPHMTFDEDRCECVCKAPCPGDLIQHPENCSCFECKESLESCCQKHKIFHPDTCRSMVFSLSP

SEQ ID NO: 11 - protein, 5 aa, Mus musculus
  MISC_FEATURE: mouse AA (D2 insertion)
DFSFE

SEQ ID NO: 12 - DNA, 649 bp, Homo sapiens
  misc_feature: VEGF-A cDNA
tcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagcgtttcgggaaccagatctctcaccagg

SEQ ID NO: 13 - protein, 191 aa, Homo sapiens
  MISC_FEATURE: VEGF-A AA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 14 - protein, 147 aa, Homo sapiens
  MISC_FEATURE: VEGF121 AA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENCDKPRR

SEQ ID NO: 15 - protein, 171 aa, Homo sapiens
  MISC_FEATURE: VEGF145 AA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 16 - protein, 191 aa, Homo sapiens; identical to SEQ ID NO: 13
  MISC_FEATURE: VEGF165 AA

SEQ ID NO: 17 - protein, 215 aa, Homo sapiens
  MISC_FEATURE: VEGF189 AA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 18 - protein, 232 aa, Homo sapiens
  MISC_FEATURE: VEGF206 AA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

(19) United States
(12) Patent Application Publication
(10) Pub. No.: US 2012/0141424 A1
(43) Pub. Date: Jun. 7, 2012

(54) Materials and Methods for the Treatment of Hypertension

(75) Inventors: Kari Alitalo, Helsinki (FI); Tuomas Tammela, Helsinki (FI); Georgia Zarkada, Helsinki (FI); Denis Tvorogov, Masala (FI)

(73) Assignee: VEGENICS PTY LIMITED, Toorak, Victoria (AU)

(21) Appl. No.: 13/143,506

(22) PCT Filed: Jan. 7, 2010
(86) PCT No.: PCT/AU10/00013

§ 371 Date: Oct. 6, 2011

Related U.S. Application Data
(60) Provisional application No. 61/142,996, filed on Jan. 7, 2009.

(51) Int. Cl. A61K 38/18; A61K 31/122; A61K 35/76; A61P 9/12; A61K 31/7088

(52) U.S. Cl. 424/93.6; 514/8.1; 514/44 R; 424/94.1; 514/690

(57) ABSTRACT

The present invention is directed to materials and methods for the treatment of hypertension and ischemia comprising administering at least one therapeutic agent selected from the group consisting of vascular endothelial growth factor-C product and vascular endothelial growth factor-D product, and optionally, when treating hypertension, a standard of care anti-hypertensive agent.

### FIELD OF THE INVENTION

The present invention provides materials and methods for the treatment of hypertension.

### BACKGROUND

All references, including any patents or patent application, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in Australia or in any other country.

Hypertension, also referred to as high blood pressure, is a medical condition in which the blood pressure is chronically elevated. Hypertension can be classified as either essential (primary) or secondary. Essential hypertension indicates that no specific medical cause can be found to explain a patient's condition. Secondary hypertension indicates that the high blood pressure is a result of (i.e. secondary to) another condition, such as kidney disease or tumors (pheochromocytoma and paraganglioma). Persistent hypertension is one of the risk factors for strokes, heart attacks, heart failure and arterial aneurysm, and is a leading cause of chronic renal failure. Even moderate elevation of arterial blood pressure leads to shortened life expectancy. At severely high pressures, defined as mean arterial pressures 50% or more above average, a person can expect to live no more than a few years unless appropriately treated.

Systolic blood pressure is the peak pressure exerted on the walls of the arteries during the contraction phase of the ventricles of the heart. Diastolic blood pressure is the minimum pressure exerted on the vessel walls when the heart muscle relaxes between beats and is filling with blood. The mean arterial blood pressure is the product of cardiac output and peripheral vascular resistance.

Pre-hypertension has been defined as a systolic blood pressure in the range of from 120 mmHg to 139 mm HG and/or a diastolic blood pressure in the range of from 80 mmHg to 89 mmHg. Pre-hypertension is considered to be a precursor of hypertension and a predictor of excessive cardiovascular risk (Julius, S., et al., N. Engl. J. Med., 354:1685-1697 (2006)).

Hypertension, or elevated BP, has been defined as a systolic blood pressure of at least 140 mmHg and/or a DBP of at least 90 mmHg. By this definition, the prevalence of hypertension in developed countries is about 20% of the adult population, rising to about 60-70% of those aged 60 or more, although a significant fraction of these hypertensive subjects have normal blood pressure when this is measured in a non-clinical setting. Some 60% of this older hypertensive population have isolated systolic hypertension, i.e. they have an elevated systolic blood pressure and a normal diastolic blood pressure. Hypertension is associated with an increased risk of stroke, myocardial infarction, atrial fibrillation, heart failure, peripheral vascular disease and renal impairment (Fagard, R H; Am. J. Geriatric Cardiology, 11(1), 23-28, 2002); Brown, M J and Haycock, S; Drugs, 59(Suppl2), 1-12, 2000).

The pathophysiology of hypertension is the subject of continuing debate. While it is generally agreed that hypertension is the result of an imbalance between cardiac output and peripheral vascular resistance, and that most hypertensive subjects have normal cardiac output and increased peripheral resistance there is uncertainty which parameter changes first (Beevers, G et al.; BMJ, 322, 912-916, 2001).

There are a number of antihypertensive drugs on the market. These drugs usually fall into the following categories: (1) diuretics (e.g., chlorothiazide, spironolactone), which cause the body to excrete water and salt; (2) β-blockers (e.g., acebutolol, atenolol, betaxolol, bisprolol, metoprolol), which block the effects of epinephrine and norepinephrine, thus easing the heart's pumping action and via indirect mechanisms relaxing blood vessels; (3) calcium channel blockers (e.g., verapamil, diltiazem, nifedipine, amlodipine, felodipine, isradipine, nicardipine, nisoldipine), which help decrease the contractions of the heart and widen blood vessels (4) ACE inhibitors (e.g., benazepril, captopril, enalapril, lisinopril, moexipril, quinapril, ramipril, trandolapril), which reduce the production of angiotensin II, a chemical that causes arteries to constrict; (5) angiotensin II receptor blockers (e.g., irbesartan, losartan, valsartan), which interfere with the renin-angiotensin system; (6) sympatholytics including centrally acting agonists (e.g., clonidine, guanabenz, guanfacine, methyldopa), α₁-adrenergic blockers (e.g., doxazosin, prazosin, terazosin,) and peripheral-acting adrenergic ganglionic blockers (e.g., guanadrel sulfate, guanethidine, reserpine); (7) vasodilators (e.g., minoxidil, hydralazine), which dilate or relax blood vessels and (8) Aliskiren.

Despite the large number of drugs available in various pharmacological categories, there is still a need in the art for new and effective treatments of hypertension.

### SUMMARY OF THE INVENTION

The present invention includes materials (molecules, compositions, kits, unit doses, etc.) and methods for therapeutic or prophylactic treatment of hypertension, and for diagnosing, evaluating, or monitoring hypertension. Similarly, the invention includes therapeutic and prophylactic uses of materials, including uses for the manufacture of medicaments for hypertension.

In one aspect, the invention provides a method of treating a mammalian subject suffering from hypertension comprising administering to said subject a composition comprising at least one therapeutic agent selected from the group consisting of a VEGF-C growth factor product and a VEGF-D growth factor product, wherein said composition is administered in an amount effective to reduce systolic or diastolic blood pressure in said subject.

In another aspect, the invention provides a method of treating hypertension in a subject, the method comprising identifying a subject as having secondary hypertension; and administering to said subject a composition comprising at least one therapeutic agent selected from the group consisting of a VEGF-C growth factor product and a VEGF-D growth factor product, wherein said composition is administered in an amount effective to reduce systolic or diastolic blood pressure in said subject. The identifying step may comprise determining whether the subject has a disorder or condition selected from the group consisting of pregnancy, cancer, polycystic kidney disease, chronic glomerulonephritis, disease of the renal arteries, aldosteronism, Cushing's syndrome and pheochromocytoma.

In yet another aspect, the invention provides a method of treating a mammalian subject suffering from hypertension comprising administering to said subject a composition comprising at least one therapeutic agent that activates a vascular endothelial growth factor selected from the group consisting of VEGF-C and VEGF-D, wherein said composition is administered in an amount effective to reduce systolic or diastolic blood pressure in said subject. In some embodiments, the therapeutic agent is a serine protease (i.e. plasmin). The plasmin may be purified.

In another aspect, the invention provides a use of a therapeutic selected from the group consisting of a VEGF-C growth factor product and a VEGF-D growth factor product in the manufacture of a medicament for the treatment of hypertension in a mammalian subject. In some embodiments, the medicament is administered in an amount effective to reduce systolic or diastolic blood pressure in said subject.

In another aspect, the invention provides a therapeutic selected from the group consisting of a VEGF-C growth factor product and a VEGF-D growth factor product in the manufacture of a medicament for the treatment of hypertension in a mammalian subject identified as having secondary hypertension. In some embodiments, the medicament is administered in an amount effective to reduce systolic or diastolic blood pressure in said subject. A subject may be identified as having secondary hypertension by determining whether the subject has a disorder or condition selected form the group consisting of pregnancy, cancer, polycystic kidney disease, chronic glomerulonephritis, disease of the renal arteries, aldosteronism, Cushing's syndrome and pheochromocytoma.

In yet another aspect, the invention provides use of at least one therapeutic agent that activates a vascular endothelial growth factor selected from the group consisting of VEGF-C and VEGF-D in the manufacture of a medicament for the treatment of hypertension in a mammalian subject. In some embodiments, the medicament is administered in an amount effective to reduce systolic or diastolic blood pressure in said subject. In some embodiments, the therapeutic agent is a serine protease (i.e. plasmin). The plasmin may be purified.

As already noted, many methods, uses, compositions of matter, and other aspects of the invention involve or include a VEGF-C or VEGF-D growth factor product. Exemplary VEGF-C or VEGF-D growth factor products (for use individually or in combination) include molecules such as VEGF-D and VEGF-C polypeptides that bind and stimulate phosphorylation of VEGFR-3 and or VEGFR-2; and polynucleotides that comprise a nucleotide sequence that encodes a VEGF-D or a VEGF-C polypeptide that binds and stimulates phosphorylation of VEGFR-3 and/or VEGFR-2. Exemplary VEGF-C or VEGF-D polypeptides comprise amino acid sequences similar or identical to the amino acid sequence of a naturally-occurring mammalian VEGF-C or VEGF-D polypeptide, with polypeptides having a sequence identical to the wildtype sequence in a subject being highly preferred. For example, human VEGF-C and VEGF-D are highly preferred for human subjects. Optionally, the VEGF-D polypeptide or VEGF-C polypeptide is attached to a heterologous polypeptide. Such heterologous constructs can be expressed as fusion proteins when the attachment is by means of a peptide bond. Numerous polynucleotides are capable of encoding any individual polypeptide due to the well-known degeneracy of the genetic code, and all are suitable for practice of the invention. Preferred polynucleotides include a wildtype VEGF-C or VEGF-D coding sequence.

Both VEGF-D and VEGF-C undergo proteolytic processing in vivo whereby a signal peptide, C-terminal pro-peptide, and N-terminal pro-peptide are cleaved to produce a fully-processed form. All forms (pre-propeptide forms, partly-processed, and fully-processed) are contemplated for practice of the invention, and the processing can be mimicked recombinantly by deletions of portion(s) of the coding sequence, such as deletion of an N-terminal pro-peptide (ΔN) and/or deletion of a C terminal propeptide (ΔC). Thus, in some variations of the invention, the VEGF-D polypeptide comprises a full length VEGF-D polypeptide, a VEGF-DΔNΔC polypeptide, a VEGF-DΔN polypeptide, and/or a VEGF-DΔC polypeptide, wherein the VEGF-D polypeptide binds and stimulates phosphorylation of VEGFR-3 and/or VEGFR-2. In some variations of the invention, the VEGF-C polypeptide comprises a full length VEGF-C polypeptide, a VEGF-CΔNΔC polypeptide, a VEGF-CΔN polypeptide, and a VEGF-CΔC polypeptide, wherein the VEGF-C polypeptide binds and stimulates phosphorylation of VEGFR-3 and/or VEGFR-2.

With respect to VEGF-C, it has been shown that mature VEGF-C is capable of binding and stimulating VEGFR-2, but removal of a cysteine residue (e.g., by substitution or deletion) abrogates this activity. The cysteine corresponds to position 156 of the wildtype human VEGF-C amino acid sequence. For all aspects of the invention described herein relating to VEGF-C, a VEGF-C ΔCys₁₅₆ polypeptide is contemplated as a variation for practicing the invention. The term “VEGF-C ΔCys₁₅₆” refers to deletion of the indicated cysteine or substitution with another amino acid. Similar terms for such molecules also are used herein, including, for example “VEGF-C C156X” or “VEGF-C156X”, in which the cysteine at position 156 is deleted or replaced with an amino acid, X, other than cysteine (for example, serine; VEGF-C156S). Preferred VEGF-C ΔCysᵢ₅₆ polypeptides bind and stimulate phosphorylation of VEGFR-3.

VEGF-C and VEGF-D growth factor products for use in the materials, methods, and uses of the invention also can be characterized by reference to structural features, such as amino acid sequence. For example, in some variations of the invention, the VEGF-C growth factor product binds and stimulates phosphorylation of VEGFR-3 (and/or VEGFR-2) and comprises a (at least one) polypeptide selected from the group consisting of:
(a) a polypeptide comprising amino acids 1-419 of SEQ ID NO: 2 or a polypeptide comprising amino acids 32-419 of SEQ ID NO: 2;
(b) a polypeptide comprising amino acids 103-227 of SEQ ID NO: 2;
(c) N-terminal deletion fragments of (a) or (b) that bind and stimulate phosphorylation of VEGFR-3 and/or VEGFR-2;
(d) C-terminal deletion fragments of (a) or (b) or (c) that bind and stimulate phosphorylation of VEGFR-3 and/or VEGFR-2;
(e) polypeptides that comprise an amino acid sequence at least 75% identical to (a) or (b) or (c) or (d) that binds and stimulates phosphorylation of VEGFR-3 and/or VEGFR-2; and
(f) polypeptides according to (a), (b), (c), (d), or (e), wherein the cysteine corresponding to position 156 of SEQ ID NO: 2 has been deleted or replaced with another amino acid and wherein the polypeptides bind and stimulate phosphorylation of VEGFR-3. With respect to (e), sequences at least 80, 85, 90, 91, 92, 93, 94, 95, 96, 97, 98, 99, and 99.5% identical are also contemplated.

Category (f) describes VEGF-C ΔC156 polypeptides. In addition, such peptides can be characterized with respect to their own exemplary amino acid sequence, such as that set forth in SEQ ID NO: 6. Thus, in some variations of the invention, the VEGF-C growth factor product comprises a (at least one) member selected from the group consisting of:
(a) a polypeptide comprising amino acids 1-419 of SEQ ID NO: 6 or a polypeptide comprising amino acids 32-419 of SEQ ID NO: 6;
(b) a polypeptide comprising amino acids 103-227 of SEQ ID NO: 6;
(c) N-terminal deletion fragments of (a) or (b) that bind and stimulate phosphorylation of VEGFR-3 and/or VEGFR-2;
(d) C-terminal deletion fragments of (a) or (b) or (c) that bind and stimulate phosphorylation of VEGFR-3 and/or VEGFR-2; and
(e) polypeptides that comprise an amino acid sequence at least 75% identical to (a) or (b) or (c) or (d), with the proviso that the amino acid corresponding to position 156 of SEQ ID NO: 6 is not cysteine and wherein the polypeptides bind and stimulate phosphorylation of VEGFR-3. Sequences at least 80, 85, 90, 91, 92, 93, 94, 95, 96, 97, 98, 99, and 99.5% identical are also contemplated.

Similarly, in some variations of the invention, the VEGF-D growth factor product binds and stimulates phosphorylation of VEGFR-3 (and/or VEGFR-2) and comprises a (at least one) polypeptide selected from the group consisting of:
(a) a polypeptide comprising amino acids 1-354 of SEQ ID NO: 4 or a polypeptide comprising amino acids 22-354 of SEQ ID NO: 4;
(b) a polypeptide comprising amino acids 93-201 of SEQ ID NO: 4;
(c) N-terminal deletion fragments of (a) or (b) that bind and stimulate phosphorylation of VEGFR-3 and/or VEGFR-2;
(d) C-terminal deletion fragments of (a) or (b) or (c) that bind and stimulate phosphorylation of VEGFR-3 and/or VEGFR-2; and
(e) polypeptides that comprise an amino acid sequence at least 75% identical to (a) or (b) or (c) or (d) that bind and stimulate phosphorylation of VEGFR-3 and/or VEGFR-2. Sequences at least 80, 85, 90, 91, 92, 93, 94, 95, 96, 97, 98, 99, and 99.5% identical are also contemplated.

In still other variations of the invention, the VEGF-C or VEGF-D growth factor product can be delivered as a polynucleotide that is transcribed and translated in vivo to produce a ligand that binds and stimulates VEGFR-3 and/or VEGFR-2. Thus, in some variations, the VEGF-C or VEGF-D growth factor product comprises a polynucleotide (nucleic acid) that comprises a nucleotide sequence that encodes any of the polypeptide VEGF-C or VEGF-D growth factor products described herein, especially the VEGF-D polypeptides and VEGF-C polypeptides described in the preceding paragraphs.

The structure of both VEGF-C and VEGF-D variant polypeptides and encoding polynucleotides for use according to the invention also can be expressed in terms of percent identity to a reference polynucleotide sequence, or by ability to hybridize to a reference polynucleotide sequence under conditions of low, medium, or high stringency conditions.

For example, in some variations of the invention, the VEGF-C or VEGF-D growth factor comprises a polynucleotide that comprises a nucleotide sequence at least 75%, 80%, 85%, 90%, 91%, 92%, 93%, 94%, 95%, 96%, 97%, 98%, or 99% identical to the sequence of SEQ ID NO: 1, or 3 or 5, and wherein the polynucleotide encodes a polypeptide that binds and stimulates phosphorylation of VEGFR-3 and/or VEGFR-2.

In some variations, the VEGF-C or VEGF-D growth factor product comprises a polynucleotide that comprises a nucleotide sequence that hybridizes to the complement of SEQ ID NO: 1 or 3 or 5 under high stringency conditions, and wherein the polynucleotide encodes a polypeptide that binds and stimulates phosphorylation of VEGFR-3 and/or VEGFR-2.

In preferred variations, the polynucleotide includes one or more sequences in addition to a coding sequence to facilitate or promote expression of the polynucleotide in cells of the subject (e.g., promoters, enhancers, etc.). Likewise, the polynucleotide is preferably packaged or formulated to facilitate uptake by target cells, e.g., using suitable gene therapy vectors, liposome, or other delivery vehicles.

Thus, in some variations of the invention the polynucleotide VEGF-C or VEGF-D growth factor product comprises an expression vector that contains the polynucleotide with its coding sequence operatively connected to at least one expression control sequence. Exemplary vectors include viral vectors, including replication-deficient viral vectors, such as adenoviral vectors, adeno-associated virus vectors, lentivirus vectors, herpes virus vectors, and vaccinia virus vectors.

With respect to all methods, uses, and materials described herein, embodiments are contemplated in which the materials are formulated with a pharmaceutically acceptable diluent, excipient, or carrier. For example, embodiments are contemplated in which a VEGF-C or VEGF-D growth factor product, alone or in combination with other active agents described herein, is combined with a pharmaceutically acceptable diluent, excipient, or carrier.

Still other embodiments of the invention involve combination therapy (methods of prophylaxis or therapy) and materials/compositions using a VEGF-C or VEGF-D growth factor product and standard of care therapeutics for the treatment of hypertension. With respect to methods/uses, the agents can be administered simultaneously or sequentially. With respect to materials, the agents can be combined in admixture or packaged together as a kit. Exemplary standard of care regimens for hypertension include, but are not limited to, Coenzyme Q10; renin inhibitors (e.g., aliskiren); angiotensin-convertin enzyme (ACE) inhibitors (e.g., captopril, enalapril maleate, ramipril, meoxipril, quinapril hydrochloride, lisinopril, benazepril hydrochloride, trandolapril and fosinopril sodium); angiotensin II receptor (AR) blockers (e.g., losartan potassium, candesartan, irbesartan, eprosartan, olmesartan, valsartan, and telmisartan), alpha blockers (e.g., doxazosin, prazosin, and terazosin); diuretics (e.g., hydrochlorothiozide, bendroflumethiazide, spironolactone, amiloride hydrochloride, triamterene, furosemide, torsemide, bumetanide, and ethacrynic acid); beta blockers (e.g., acebutolol, bisoprolal, carteolol, carvedilo, celiprolol, labetalol, mepindolol, metoprolol, oxprenolol, pindolol, atenolol, celiprolol, nadolol, nebivolol, sotalol, timolol, betaxolol, propranolol, and carvedilol); and calcium channel blockers (e.g., amlodipine besylate, felodipine, isradipine, nicardipine, nifedipine, nimodipine, nisoldipine, nitrendipine, lacidipine, lercanidipine, verapamil hydrochloride, gallopamil, and diltiazem hydrochloride).

Still other embodiments of the invention involve combination therapy (methods of prophylaxis or therapy) and materials/compositions using a VEGF-C or VEGF-D growth factor product and a VEGF growth factor product for the treatment of hypertension or acute ischemia. Exemplary VEGF growth factor products include a molecule such as VEGF-A polypeptides (including its various isoforms described in more detail herein) that bind and stimulate phosphorylation of VEGFR-1 and/or VEGFR-2; and polynucleotides that comprise a nucleotide sequence that encodes a VEGF-A polypeptide that binds and stimulates phosphorylation of VEGFR-1 and/or VEGFR-2.

Still other embodiments of the invention involve a method of treating hypertension in a subject in need thereof comprising identifying a subject as being resistant to treatment with a standard of care anti-hypertensive agent and administering a VEGF-C and or VEGF-D growth factor product to the subject. A subject is considered resistant to treatment when the blood pressure of a subject remains elevated above treatment goals (target normotensive pressure) despite administration of a three drug treatment regimen that includes a diuretic (Nuesch et al., British Medical Journal, 323:12-146, 2001).

Another aspect of the invention related to a method of treating acute ischemia in a subject. The method comprises administering to the subject a composition comprising at least one therapeutic agent selected from the group consisting of a VEGF-C growth factor product and a VEGF-D growth factor product, wherein the composition is administered in an amount effective to treat acute ischemia in the subject. In some embodiments, the composition is administered locally to an ischemic tissue or organ. In some embodiments, the acute ischemic is myocardial ischemia. The VEGF-C and VEGF-D growth factor products contemplated for treatment of ischemia are the same as those contemplated for treatment of hypertension, and the description of such products herein is intended to be applicable to both indications. Co-therapy with other anti-ischemia agents or with VEGF-A is contemplated as a variation of the invention.

Use of the therapeutic VEGF-C and/or VEGF-D growth factor products (or a compositions comprising the VEGF-C and/or VEGF-D growth factor products) in the manufacture of a medicament for the treatment of hypertension is also contemplated as an aspect of the invention.

Use of the therapeutic VEGF-C and/or VEGF-D growth factor products (or a compositions comprising the VEGF-C and/or VEGF-D growth factor products) in the manufacture of a medicament for the treatment of acute ischemia is also contemplated as aspect of the invention.

Additional aspects, features and variations of the invention will be apparent from the entirety of this application, including the detailed description, and all such features are intended as aspects of the invention. It should be understood, however, that the detailed description and the specific examples are given by way of illustration, and that the many various changes and modifications that will be apparent to those familiar with the field of the invention are also part of the invention.

In the claims of this application and in the description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention and to include the elements explicitly listed, and optionally, additional elements.

Aspects of the invention described with “a” or “an” should be understood to include “one or more” unless the context clearly requires a narrower meaning.

Moreover, features of the invention described herein can be re-combined into additional embodiments that also are intended as aspects of the invention, irrespective of whether the combination of features is specifically mentioned above as an aspect or embodiment of the invention. Also, only those limitations that are described herein as critical to the invention should be viewed as such; variations of the invention lacking features that have not been described herein as critical are intended as aspects of the invention.

With respect to aspects of the invention that have been described as a set or genus, every individual member of the set or genus is intended, individually, as an aspect of the invention, even if, for brevity, every individual member has not been specifically mentioned herein. When aspects of the invention that are described herein as being selected from a genus, it should be understood that the selection can include mixtures of two or more members of the genus. Similarly, with respect to aspects of the invention that have been described as a range, such as a range of values, every sub-range within the range is considered an aspect of the invention.

In addition to the foregoing, the invention includes, as an additional aspect, all embodiments of the invention narrower in scope in any way than the variations specifically described herein. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE FIGURES

FIG. 1 shows that VEGF-C decreased systolic blood pressure in mice.

FIG. 2 shows the fold change in blood pressure levels 2 minutes following intravenous injection of rhVEGF-CΔNΔC at indicated doses.

FIG. 3 demonstrates the stimulation of VEGFR-3 induced phosphorylation of eNOS in porcine aortic endothelial cells. (a) PAE-Flt4 and PAE-KDR cells expressed exclusively human VEGFR-3 and human VEGFR-2 respectively. (b) VEGF-C induced eNOS phosphorylation (arrows) via VEGFR-3 in PAE-Flt4 cells. (c) VEGF-A activation (arrowheads) induced eNOS phosphorylation (arrows) via VEGFR-2 (PAE-KDR cells) and served as a positive control.

FIG. 4 demonstrates the stimulation of human dermal microvascular endothelial cells (HDMECs) with VEGF-C induced phosphorylation of eNOS. After overnight incubation with 0.5% FBS media, HDMECs were stimulated for 15 minutes with VEGF-A (V-A), VEGF-C (V-C) or a combination of VEGF-A and VEGF-C (V-A+V-C). Subsequently, lysates were blotted with (A) phospho-eNOS and eNOS antibodies or (B) phospho-AKT and AKT antibodies.

### DETAILED DESCRIPTION OF THE INVENTION

The present invention describes materials and methods for the treatment of hypertension.

An estimated 600 million people worldwide suffer from hypertension (Cardiovascular Diseases—Prevention and Control, WHO, 2001-2002). If untreated, it carries a high mortality. Risk factors for hypertension include family history, race (most common in blacks), stress, obesity, a diet high in saturated fats or sodium, tobacco use, sedentary lifestyle, and aging. The adequate treatment of hypertension has been adamantly shown to reduce co-morbidity, such as stroke, intracerebral hemorrhage, myocardial infarction, heart failure, and kidney failure.

Water molecules filtrate continuously from the arterial side of the capillary bed into the interstitial space. Approximately 90% of the extravasated water is reabsorbed at the venous side of the capillary bed, where the colloid osmotic pressure of the blood exceeds blood pressure, but the remaining 10% results in a net excess of protein-rich fluid in the interstitial space. The main function of the lymphatic vasculature is to return this excess fluid back to the blood circulation system. Fluid, macromolecules, and cells enter blind-ended lymphatic capillaries in tissues. The lymph is further transported towards collecting lymphatic vessels and is returned to the blood circulation through the lymphatico-venous junctions at the subclavian veins. Lymphatic vessels are generally considered solely as passive means to return extracellular fluid to the central circulation. However, their role in the pathogenesis of hypertension has not been previously considered.

Vascular endothelial growth factors (VEGFs) stimulate angiogenesis and lymphangiogenesis by activating VEGF receptor (VEGFR) tyrosine kinases in endothelial cells (Carmeliet et al., Nature, 438:936-936, 2005). VEGFR-3 is present on all endothelia during development, but in the adult its expression becomes primarily restricted to the lymphatic endothelium (Kaipainen et al., Proc Natl Acad Sci USA. 1995, 92:3566-70). VEGF-C and VEGF-D are ligands for VEGFR-3, and primarily induce lymphangiogenesis in adult tissues (Alitalo et al., Nature, 2005).

VEGF-C also has direct effects on the blood vascular endothelium and hemodynamics: The processed form of VEGF-C binds to VEGFR-2 (Joukov et al., J Biol Chem, 1996), which leads to increased nitric oxide synthesis via activation of endothelial nitric oxide synthase (eNOS), as well as increased eNOS biosynthesis (Kroll and Waltenberger, Biochem Biophys Res Commun, 1998; Kroll and Waltenberger, Biochem Biophys Res Commun, 1999).

### I. VEGF-C AND VEGF-D GROWTH FACTOR PRODUCTS

The invention contemplates the use of VEGF-C or VEGF-D growth factor products for use in the methods described herein for the treatment of hypertension. Exemplary VEGF-C or VEGF-D growth factor products include VEGF-C, VEGF-CΔC₁₅₆ and VEGF-D polypeptides and polynucleotides encoding said polypeptides.

VEGF-C, VEGF-D and VEGFR-3 are members of a complex network of growth factors and receptors involved in several areas of development known as the PDGF/VEGF and PDGFR/VEGFR family proteins. The PDGF subfamily is reviewed in Heldin et al., Biochimica et Biophysica Acta 1378:F79-113 (1998).

### A. VEGF-C Growth Factor Products

In some embodiments, the VEGF-C growth factor product comprises a VEGF-C polypeptide that binds and stimulates phosphorylation of VEGFR-3 and/or VEGFR-2. VEGF-C is involved in the regulation of lymphangiogenesis: when VEGF-C was overexpressed in the skin of transgenic mice, a hyperplastic lymphatic vessel network was observed, suggesting that VEGF-C induces lymphatic growth (Jeltsch et al., Science, 276:1423-1425, 1997). Continued expression of VEGF-C in the adult also indicates a role in maintenance of differentiated lymphatic endothelium [Ferrara, J Mol Med 77:527-543 (1999)]. VEGF-C also shows angiogenic properties: it can stimulate migration of bovine capillary endothelial (BCE) cells in collagen and promote growth of human endothelial cells (see, e.g., U.S. Pat. No. 6,245,530; U.S. Pat. No. 6,221,839; and International Patent Publication No. WO 98/33917, incorporated herein by reference in their entireties).

VEGF-C (SEQ ID NOs: 1 and 2) was isolated from conditioned media of PC-3 prostate adenocarcinoma cell line (CRL1435) by selecting for a component of medium that caused tyrosine phosphorylation of the endothelial cell-specific receptor tyrosine kinase Flt4, using cells transfected to express Flt4. VEGF-C was purified using affinity chromatography with recombinant Flt4, and was cloned from a PC-3 cDNA library. Its isolation and characteristics are described in detail in Joukov et al, EMBO J. 15:290-298, 1996, and U.S. Pat. Nos. 6,221,839; 6,235,713; 6,361,946; 6,403,088; and 6,645,933 and International Patent Publ. Nos. WO 97/05250, WO 98/07832, and WO 98/01973, all of which are incorporated herein by reference in their entireties.

VEGF-C is originally expressed as a larger precursor protein, prepro-VEGF-C, having extensive amino- and carboxy-terminal peptide sequences flanking a VEGF homology domain (VHD), with the C-terminal peptide containing tandemly repeated cysteine residues in a motif typical of Balbiani ring 3 protein. The prepro-VEGF-C polypeptide is processed in multiple stages to produce a mature and most active VEGF-C polypeptide (VEGF-C ΔNΔC, residues 103-227 of SEQ ID NO: 2) of about 21-23 kD (as assessed by SDS-PAGE under reducing conditions). Such processing includes cleavage of a signal peptide (residues 1-31 of SEQ ID NO: 2); cleavage of a carboxyl-terminal peptide (corresponding approximately to amino acids 228-419 of SEQ ID NO: 2 to produce a partially-processed form of about 29 kD; and cleavage (apparently extracellularly) of an amino-terminal peptide (corresponding approximately to amino acids 32-102 of SEQ ID NO: 2) to produced a fully-processed mature form of about 21-23 kD. Experimental evidence demonstrates that partially-processed forms of VEGF-C (e.g., the 29 kD form) are able to bind the Flt4 (VEGFR-3) receptor, whereas high affinity binding to VEGFR-2 occurs only with the fully processed forms of VEGF-C. Moreover, it has been demonstrated that amino acids 103-227 of SEQ ID NO: 2 are not all critical for maintaining VEGF-C functions. A polypeptide consisting of amino acids 112-215 (and lacking residues 103-111 and 216-227) of SEQ ID NO: 2 retains the ability to bind and stimulate VEGF-C receptors, and it is expected that a polypeptide spanning from about residue 131 to about residue 211 of SEQ ID NO: 2 will retain VEGF-C biological activity. It appears that VEGF-C polypeptides naturally associate as non-disulfide linked dimers.

An alignment of human VEGF-C with VEGF-C from other species (performed using any generally accepted alignment algorithm) suggests additional residues wherein modifications can be introduced (e.g., insertions, substitutions, and/or deletions) without destroying VEGF-C biological activity. Any position at which aligned VEGF-C polypeptides of two or more species have different amino acids, especially different amino acids with side chains of different chemical character, is a likely position susceptible to modification without concomitant elimination of function. An exemplary alignment of human, murine, and quail VEGF-C is set forth in FIG. 5 of PCT/US98/01973.

Apart from the foregoing considerations, it will be understood that innumerable conservative amino acid substitutions can be performed to a wildtype VEGF-C polypeptide sequence which are likely to result in a polypeptide that retains VEGF-C biological activities, especially if the number of such substitutions is small. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine). Addition or deletion of one or a few internal amino acids without destroying VEGF-C biological activities also is contemplated.

Candidate VEGF-C analog polypeptides can be rapidly screened first for their ability to bind and stimulate autophosphorylation of known VEGF-C receptors (VEGFR-2 and VEGFR-3). Polypeptides that stimulate one or both known receptors are rapidly re-screened in vitro for their mitogenic and/or chemotactic activity against cultured capillary or arterial endothelial cells (e.g., as described in WO 98/33917). Polypeptides with mitogenic and/or chemotactic activity are then screened in vivo as described herein for efficacy in methods of the invention. In this way, variants (analogs) of naturally occurring VEGF-C proteins are rapidly screened to determine whether or not the variants have the requisite biological activity to constitute “VEGF-C polypeptides” for use in the present invention.

In some embodiments, the VEGF-C growth factor product is a VEGF-C polypeptide that selectively binds VEGFR-3. By “selectively binds VEGFR-3” is meant that the polypeptide fails to significantly bind VEGFR-2 and is not proteolytically processed in vivo into a form that shows significant reactivity with VEGFR-2. An exemplary VEGFR-3 specific VEGF-C polypeptide comprises a VEGF-C156X polypeptide (See SEQ ID NO: 6 and corresponding nucleotide sequence in SEQ ID NO: 5), in which the cysteine at position 156 is deleted or replaced with an amino acid, X, other than cysteine (for example, serine; VEGF-C156S) (see, U.S. Pat. Nos. 6,130,071 and 6,361,946; and International Patent Publication No. WO 98/33917, the disclosures of which are incorporated herein by reference in their entireties). By “VEGF-C156X polypeptide” or “VEGF-CΔC₁₅₆ polypeptide” is meant an analog wherein the cysteine at position 156 of SEQ ID NO: 2 has been deleted or replaced by another amino acid. A VEGF-C156X polypeptide analog can be made from any VEGF-C polypeptide of the invention that comprises all of SEQ ID NO: 4 or a portion thereof that includes position 156 of SEQ ID NO: 2. Preferably, the VEGF-C156X polypeptide analog comprises a portion of SEQ ID NO: 2 effective to permit binding to VEGFR-3 and has reduced VEGFR-2 binding affinity. Proteolytic processing of the exemplary sequence for a prepro-VEGF-C156X (SEQ ID NOs: 5 and 6) is the same as described above for VEGF-C, and the same portions are contemplated as active fragments.

In some embodiment, the VEGF-C growth factor product is a VEGF-C polypeptide that binds VEGFR-3 but has reduced VEGFR-2 binding affinity (e.g., VEGF-C ΔR₂₂₆ΔR₂₂₇ polypeptides). See, U.S. Pat. No. 6,130,071, the disclosure of which is incorporated herein by reference in its entirety. By “VEGF-C ΔR₂₂₆ΔR₂₂₇ polypeptide” is meant an analog wherein the arginine residues at positions 226 and 227 of SEQ ID NO: 2 have been deleted or replaced by other amino acids, for the purpose of eliminating a proteolytic processing site of the carboxy terminal pro-peptide of VEGF-C. Preferably, the VEGF-C ΔR₂₂₆ΔR₂₂₇ polypeptide comprises a portion of SEQ ID NO: 2 effective to permit binding of VEGFR-3. For example, the invention includes a VEGF-C ΔR₂₂₆ΔR₂₂₇ polypeptide having an amino acid sequence comprising amino acids 112-419 of SEQ ID NO: 2, wherein the arginine residues at positions 226 and 227 of SEQ ID NO: 2 have been deleted or replaced (e.g., with a serine residue).

In one aspect, VEGF-C polypeptides for use in a method described herein comprise an amino acid sequence at least 80%, 85%, 90%, 91%, 92%, 93%, 94%, 95%, 96%, 97%, 98%, 99%, 99.5%, or more identical to a polypeptide selected from the group consisting of (a) a polypeptide comprising amino acids 1-419 of SEQ ID NO: 2 or a polypeptide comprising amino acids 32-419 of SEQ ID NO: 2; (b) a polypeptide comprising amino acids 103-227 of SEQ ID NO: 2, (c) a polypeptide comprising amino acids 131-211 of SEQ ID NO: 2; (d) a polypeptide comprising amino acids 32-227 of SEQ ID NO: 2; (e) a polypeptide comprising amino acids 103-419 of SEQ ID NO: 2; and (f) polypeptides according to (a)-(e), wherein a cysteine corresponding to position 156 of SEQ ID NO: 2 has been deleted or replaced with another amino acid, wherein the polypeptides of (a)-(f) bind and stimulate phosphorylation of VEGFR-3. In another aspect, a VEGF-C polypeptide for use in a method described herein is selected from the group consisting of (a) a polypeptide comprising amino acids 1-419 of SEQ ID NO: 2 or a polypeptide comprising amino acids 32-419 of SEQ ID NO: 2; (b) a polypeptide comprising amino acids 103-227 of SEQ ID NO: 2, (c) a polypeptide comprising amino acids 131-211 of SEQ ID NO: 2; (d) a polypeptide comprising amino acids 32-227 of SEQ ID NO: 2; (e) a polypeptide comprising amino acids 103-419 of SEQ ID NO: 2; and (0 polypeptides according to (a)-(e), wherein a cysteine corresponding to position 156 of SEQ ID NO: 2 has been deleted or replaced with another amino acid, wherein the polypeptides bind and stimulate phosphorylation of VEGFR-3. Amino- or carboxy-terminal (or both) deletion fragments of any of the foregoing are also contemplated as VEGF-C growth factor products, as are conservative substitution variants, so long as the fragment or variant binds and stimulates phosphorylation of VEGFR-3 and/or VEGFR-2.

In another aspect, a VEGF-C polypeptide for use in a method described herein comprises an amino acid sequence encoded by a nucleic acid sequence at least 80%, 85%, 90%, 91%, 92%, 93%, 94%, 95%, 96%, 97%, 98%, 99%, 99.5%, or more identical to the nucleic acid sequence set forth in SEQ ID NO: 1 or SEQ ID NO: 5, wherein the encoded polypeptide binds and stimulates phosphorylation of VEGFR-3 and/or VEGFR-2.

### B. VEGF-D Growth Factor Products

VEGF-D is structurally and functionally most closely related to VEGF-C. Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-D ΔNΔC, is described in International Patent Publication No. WO 98/07832, incorporated herein by reference in its entirety. VEGF-D sequences from other species also have been reported. See Genbank Accession Nos. D89628 (Mus musculus); and AF014827 (Rattus norvegicus), for example, incorporated herein by reference.

VEGF-D, as well as human sequences encoding VEGF-D, and VEGF-D variants and analogs, have been described in detail in International Publication Number WO 98/07832; in U.S. Pat. No. 6,235,713; and in Achen, et al., Proc. Nat'l Acad. Sci. U.S.A., 95(2):548-553 (1998), all of which are incorporated herein by reference in the entirety. VEGF-D (SEQ ID NOs: 3 and 4) was isolated as an incomplete fragment from a human breast cDNA library, commercially available from Clontech, by screening with an expressed sequence tag obtained from a human cDNA library designated “Soares Breast 3NbHBst” as a hybridization probe (Achen et al., Proc. Natl. Acad. Sci. USA 95: 548-553, 1998). Full length VEGF-D was subsequently cloned from a human lung cDNA library. Its isolation and characteristics are described in detail in International Patent Application No. PCT/US97/14696 (WO 98/07832), incorporated herein by reference in its entirety.

The prepro-VEGF-D polypeptide has a putative signal peptide of 21 amino acids (residues 1-21 of SEQ ID NO: 4) and is apparently proteolytically processed in a manner analogous to the processing of prepro-VEGF-C. A “recombinantly matured” VEGF-D, VEGF-D ΔNΔC, containing amino acid residues 93-201 of SEQ ID NO: 4, and lacking residues 1-92 and 202-354 of SEQ ID NO: 4 retains the ability to activate receptors VEGFR-2 and VEGFR-3, and appears to associate as non-covalently linked dimers.

The predominant intracellular form of human VEGF-D is a homodimeric propeptide that consists of the VEGF/PDGF Homology Domain (VHD) and the N- and C-terminal propeptides. After secretion, this polypeptide is proteolytically cleaved (Stacker et al., J Biol Chem 274:32127-32136, 1999). The human VEGF-D VHD consists of residues 93-201 of full length VEGF-D and binds both VEGFR-2 and VEGFR-3.

The description of the cloning of the mouse homolog of VEGF-D is also found in International Patent Application PCT/US97/14696 (WO 98/07832). It was found that there are two isoforms of mouse VEGF-D. The longer amino acid sequence is designated mVEGF-D1, and the shorter sequence is designated mVEGF-D2. The nucleotide sequences of the cDNAs encoding mVEGF-D1 and mVEGF-D2 are found in SEQ ID NOs: 7 and 9, respectively. The deduced amino acid sequences for mVEGF-D1 and mVEGF-D2 are found in SEQ ID NOs: 8 and 10, respectively. The differences between the mVEGF-D1 and mVEGF-D2 amino acid sequences are:

i) an insertion of five amino acids (DFSFE) (SEQ ID NO: 11) after residue 30 in mVEGF-D1 in comparison to mVEGF-D2; and
ii) complete divergence of the C-terminal ends after residue 317 in mVEGF-D1 and residue 312 in mVEGF-D2, which results in mVEGF-D1 being considerably longer.

VEGF-D is highly conserved between mouse and man. 85% of the amino acid residues of human VEGF-D are identical in mouse VEGF-D1. It is also predicted that the predominant intracellular form of mouse VEGF-D is a homodimeric propeptide that consists of the VEGF/PDGF Homology Domain (VHD) and the N- and C-terminal propeptides. The mouse VHD consists of residues 92-201 of the full-length mouse VEGF-D2 (SEQ ID NO: 10).

In some embodiments, the VEGF-D growth factor product is a VEGF-D polypeptide that has been modified at either the N- or C-terminal proteolytic processing sites (e.g., VEGF-D R85SR88S, VEGF-D R204SR205S) to prevent proteolytic processing the wildtype VEGF-D polypeptide. See, International Patent Publication No. WO 2007/038056, the disclosure of which is incorporated herein by reference in its entirety. Proteolytic processing sites in VEGF-D are known in the art. See, e.g., Stacker et al. J. Biol. Chem., 274 (1999) pp. 32127-32136, the disclosure of which are incorporated herein by reference in its entirety.

In some embodiments, VEGF-D polypeptides for use in a method described herein comprise an amino acid sequence at least 80%, 85%, 90%, 91%, 92%, 93%, 94%, 95%, 96%, 97%, 98%, 99%, 99.5%, or more identical to a polypeptide selected from the group consisting of (a) a polypeptide comprising amino acids 1-354 of SEQ ID NO: 4 or a polypeptide comprising amino acids 22-354 of SEQ ID NO: 4; (b) a polypeptide comprising amino acids 93-201 of SEQ ID NO: 4, (c) a polypeptide comprising amino acids 92-354 of SEQ ID NO: 4; (d) a polypeptide comprising amino acids 22-354 of SEQ ID NO: 4; and (e) a polypeptide comprising amino acids 92-201 of SEQ ID NO: 10, wherein the polypeptides of (a)-(e) bind and stimulate phosphorylation of VEGFR-3. In another aspect, a VEGF-D polypeptide for use in a method described herein is selected from the group consisting of (a) a polypeptide comprising amino acids 1-354 of SEQ ID NO: 4 or a polypeptide comprising amino acids 22-354 of SEQ ID NO: 4; (b) a polypeptide comprising amino acids 93-201 of SEQ ID NO: 4, (c) a polypeptide comprising amino acids 92-354 of SEQ ID NO: 4; (d) a polypeptide comprising amino acids 22-354 of SEQ ID NO: 4; and (e) a polypeptide comprising amino acids 92-201 of SEQ ID NO: 10. Amino- or carboxy-terminal (or both) deletion fragments of ant of the foregoing are also contemplated as VEGF-C or VEGF-D growth factor products, as are conservative substitution variants, so long as the fragment or variant binds and stimulates phosphorylation of VEGFR-3 and/or VEGFR-2.

In some embodiments, VEGF-D polypeptides for use in a method described herein comprises an amino acid sequence encoded by a nucleic acid sequence at least 80%, 85%, 90%, 91%, 92%, 93%, 94%, 95%, 96%, 97%, 98%, 99%, 99.5%, or more identical to the nucleic acid sequence set forth in SEQ ID NO: 3, 7 or 9, wherein the encoded polypeptide binds and stimulates phosphorylation of VEGFR-3 and/or VEGFR-2.

### II. VEGF-C AND VEGF-D GROWTH FACTOR PRODUCT VARIANTS AND DERIVATIVES

The VEGF-C and VEGF-D growth factor products for use in a method of the invention can readily be modified by techniques well-known to one of ordinary skill in the art. Potential mutations include insertion, deletion or substitution of one or more residues. The term “VEGF-C polypeptide variant” refers to a VEGF-C polypeptide sequence that contains at least one amino acid substitution, deletion, or insertion in the VEGF-C wild-type amino acid sequence, wherein the variant retains the biological activity of wild-type VEGF-C.

The term “VEGF-D polypeptide variant” refers to a VEGF-D polypeptide sequence that contains at least one amino acid substitution, deletion, or insertion in the VEGF-D wild-type amino acid sequence, wherein the variant retains the biological activity of wild-type VEGF-D.

The term “VEGF-C polypeptide medication modification” when used herein includes but is not limited to, one or more amino acid change (including substitutions, insertions or deletions); chemical modifications that do not interfere with binding of a VEGF-C polypeptide to VEGFR-3 and/or VEGFR-2; covalent modification by conjugation to therapeutic or diagnostic agents; labeling (e.g., with radionuclides or various enzymes); covalent polymer attachment such as pegylation (derivatization with polyethylene glycol) and insertion or substitution by chemical synthesis of non-natural amino acids. In some embodiments, modified polypeptides of the invention will retain the binding properties of unmodified molecules of the invention.

The term “VEGF-D polypeptide medication modification” when used herein includes but is not limited to, one or more amino acid change (including substitutions, insertions or deletions); chemical modifications that do not interfere with binding of a VEGF-D polypeptide to VEGFR-3 and/or VEGFR-2; covalent modification by conjugation to therapeutic or diagnostic agents; labeling (e.g., with radionuclides or various enzymes); covalent polymer attachment such as pegylation (derivatization with polyethylene glycol) and insertion or substitution by chemical synthesis of non-natural amino acids. In some embodiments, modified polypeptides of the invention will retain the binding properties of unmodified molecules of the invention.

The term “VEGF-C polypeptide derivative” refers to VEGF-C polypeptides that are covalently modified by conjugation to therapeutic or diagnostic agents, labeling (e.g., with radionuclides or various enzymes), covalent polymer attachment such as pegylation (derivatization with polyethylene glycol) and insertion or substitution by chemical synthesis of non-natural amino acids. In some embodiments, derivatives of the invention will retain the binding properties of underivatized molecules of the invention.

The term “VEGF-D polypeptide derivative” refers to VEGF-D polypeptides that are covalently modified by conjugation to therapeutic or diagnostic agents, labeling (e.g., with radionuclides or various enzymes), covalent polymer attachment such as pegylation (derivatization with polyethylene glycol) and insertion or substitution by chemical synthesis of non-natural amino acids. In some embodiments, derivatives of the invention will retain the binding properties of underivatized molecules of the invention.

Deletion variants are polypeptides wherein at least one amino acid residue of any amino acid sequence is removed. Deletions can be effected at one or both termini of the protein, or with removal of one or more residues within (i.e. internal to) the polypeptide. Methods for preparation of deletion variants are routine in the art. See, e.g., Sambrook et al. (1989) Molecular Cloning: A Laboratory Guide, Vols 1-3, Cold Spring Harbor Press, the disclosure of which is incorporated herein by reference in its entirety.

Amino acid sequence insertions include amino- and/or carboxyl-terminal fusions ranging in length from one residue to polypeptides containing hundreds or more residues, as well as internal sequence insertions of one or more amino acids. As with any of the different variant types described herein, insertional variants can be designed such that the resulting polypeptide retains the same biological properties or exhibits a new physical, chemical and/or biological property not associated with the parental polypeptide from which it was derived. Methods for preparation of insertion variants are also routine and well known in the art (Sambrook et al., supra).

Fusion proteins comprising a VEGF-C or VEGF-D growth factor product, and a heterologous polypeptide, are a specific type of insertion variant contemplated by the invention. Non-limiting examples of heterologous polypeptides which can be fused to polypeptides of interest include anti-hypertensive agents (such as the anti-hypertensive agents described herein), proteins with long circulating half-life, such as, but not limited to, immunoglobulin constant regions (e.g., Fc region); marker sequences that permit identification of the polypeptide of interest; sequences that facilitate purification of the polypeptide of interest; and sequences that promote formation of multimeric proteins. In some embodiments, a receptor fragment is fused to alkaline phosphatase (AP). Methods for making Fc or AP fusion constructs are found in WO 02/060950.

Methods of making antibody fusion proteins are well known in the art. See, e.g., U.S. Pat. No. 6,306,393, the disclosure of which is incorporated herein by reference in its entirety. In certain embodiments of the invention, fusion proteins are produced which may include a flexible linker, which connects the chimeric scFv antibody to the heterologous protein moiety. Appropriate linker sequences are those that do not affect the ability of the resulting fusion protein to be recognized and bind the epitope specifically bound by the V domain of the protein (see, e.g., WO 98/25965, the disclosure of which is incorporated herein by reference in its entirety).

Substitution variants are those in which at least one residue in the polypeptide amino acid sequence is removed and a different residue is inserted in its place. Modifications in the biological properties of the VEGF-C or VEGF-D growth factor product are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. In certain embodiments of the invention, substitution variants are designed, i.e. one or more specific (as opposed to random) amino acid residues are substituted with a specific amino acid residue. Typical changes of these types include conservative substitutions and/or substitution of one residue for another based on similar properties of the native and substituting residues.

Conservative substitutions are shown in Table 1. The most conservative substitution is found under the heading of “preferred substitutions.” If such substitutions result in no change in biological activity, then more substantial changes may be introduced and the products screened.

TABLE 1

| | | Preferred |
|---|---|---|
| | | Residue |
| Original | Exemplary | Substitutions |
| Ala (A) | val; leu; ile | val |
| Arg (R) | lys; gln; asn | lys |
| Asn (N) | gln; his; asp, lys; gln | gln |
| Asp (D) | glu; asn | glu |
| Cys (C) | ser; ala | ser |
| Gln (Q) | asn; glu | asn |
| Glu (E) | asp; gln | asp |
| Gly (G) | ala | |
| His (H) | asn; gln; lys; arg | |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | |
| Pro (P) | ala | |
| Ser (S) | thr | |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Amino acid residues which share common side-chain properties are often grouped as follows.
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

### III. POLYNUCLEOTIDES ENCODING VEGF-C AND VEGF-D GROWTH FACTOR PRODUCTS

The invention embraces polynucleotides that encode the polypeptides of the invention. Also provided are polynucleotides that hybridize under moderately stringent or high stringency conditions to the complete non-coding strand, or complement, of such polynucleotides. Complementary molecules are useful as templates for synthesizing coding molecules, and for making stable double-stranded polynucleotides. Due to the well-known degeneracy of the universal genetic code, one can synthesize numerous polynucleotide sequences that encode each chimeric polypeptide of the present invention. All such polynucleotides are contemplated as part of the invention. Such polynucleotides are useful for recombinant expression of polypeptides of the invention in vivo or in vitro (e.g., for gene therapy).

This genus of polynucleotides embraces polynucleotides that encode polypeptides with one or a few amino acid differences (additions, insertions, or deletions) relative to amino acid sequences specifically depicted herein. Such changes are easily introduced by performing site directed mutagenesis, for example.

A genus of similar polypeptides can alternatively be defined by the ability of encoding polynucleotides to hybridize to the complement of a nucleotide sequence that corresponds to the cDNA sequence encoding the polypeptide. For example, the invention provides a polynucleotide that comprises a nucleotide sequence that hybridizes under moderately stringent or high stringency hybridization conditions to the complement of any specific nucleotide sequence of the invention, and that encodes a VEGF-C or VEGF-D growth factor product as described herein that binds and stimulates phosphorylation of VEGFR-3 and/or VEGFR-2. In one aspect, the invention provides a VEGF-D polypeptide, a VEGF-C polypeptide or a VEGF-CΔC₁₅₆ polypeptide comprising an amino acid sequence encoded by a nucleic acid sequence that hybridizes to the complement of (the coding portion of) SEQ ID NOs: 1, 3 or 5, respectively, under moderately or highly stringent conditions. In another aspect, the invention provides a VEGF-D polynucleotide, a VEGF-C polynucleotide or a VEGF-CΔC₁₅₆ polynucleotide that comprises a nucleic acid sequence that hybridizes to the complement of (the coding portion of) SEQ ID NOs: 1, 3 or 5, respectively, under moderately or highly stringent conditions.

The term “highly stringent conditions” refers to hybridization/wash conditions selected to only permit hybridization of DNA strands whose sequences are highly complementary, and to exclude hybridization of significantly mismatched DNAs. Hybridization stringency is principally determined by temperature, ionic strength, and the concentration of denaturing agents such as formamide. Exemplary highly stringent hybridization conditions are as follows: hybridization at 65° C. for at least 12 hours in a hybridization solution comprising 5×SSPE, 5×Denhardt's, 0.5% SDS, and 2 mg sonicated non homologous DNA per 100 ml of hybridization solution; washing twice for 10 minutes at room temperature in a wash solution comprising 2×SSPE and 0.1% SDS; followed by washing once for 15 minutes at 65° C. with 2×SSPE and 0.1% SDS; followed by a final wash for 10 minutes at 65° C. with 0.1×SSPE and 0.1% SDS. Moderate stringency washes can be achieved by washing with 0.5×SSPE instead of 0.1×SSPE in the final 10 minute wash at 65° C. Low stringency washes can be achieved by using 1×SSPE for the 15 minute wash at 65° C., and omitting the final 10 minute wash. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook et al., (Eds.), Molecular Cloning: A Laboratory Manual, Cold Spring Harbor Laboratory Press: Cold Spring Harbor, N.Y. (1989), pp. 9.47 to 9.51.

In some embodiments, the invention provides a polynucleotide that comprises a nucleotide sequence that is at least 75%, 80%, 85%, 90%, 91%, 92%, 93%, 94%, 95%, 96%, 97%, 98%, 99% or more identical to any specific nucleotide sequence of the invention, and that encodes a VEGF-C or VEGF-D growth factor product as described herein that binds and stimulates phosphorylation of VEGFR-3 and/or VEGFR-2. For example, in one aspect, the invention provides a polynucleotide that comprises a nucleic acid sequence at least 80%, 85%, 90%, 91%, 92%, 93%, 94%, 95%, 96%, 97%, 98%, 99%, 99.5%, or more identical to a nucleic acid sequence set forth in SEQ ID NO: 1. In another aspect, the invention provides a polynucleotide that comprises a nucleic acid sequence at least 80%, 85%, 90%, 91%, 92%, 93%, 94%, 95%, 96%, 97%, 98%, 99%, 99.5%, or more identical to a nucleic acid sequence set forth in SEQ ID NO: 3. In another aspect, the invention provides a polynucleotide that comprises a nucleic acid sequence at least 80%, 85%, 90%, 91%, 92%, 93%, 94%, 95%, 96%, 97%, 98%, 99%, 99.5%, or more identical to a nucleic acid sequence set forth in SEQ ID NO: 5.

Also provided is a polynucleotide comprising a nucleic acid sequence that encodes a polypeptide comprising an amino acid sequence at least 80%, 85%, 90%, 91%, 92%, 93%, 94%, 95%, 96%, 97%, 98%, 99%, 99.5%, or more identical to an amino acid sequence selected from the group consisting of (a) amino acids 1-419 of SEQ ID NO: 2 or amino acids 32-419 of SEQ ID NO: 2; (b) amino acids 103-227 of SEQ ID NO: 2, (c) amino acids 131-211 of SEQ ID NO: 2; (d) amino acids 32-227 of SEQ ID NO: 2; (e) amino acids 103-419 of SEQ ID NO: 2; and (f) polypeptides according to (a)-(e), wherein a cysteine corresponding to position 156 of SEQ ID NO: 2 has been deleted or replaced with another amino acid, wherein the polypeptides of (a)-(e) bind and stimulate phosphorylation of VEGFR-3 and/or VEGFR-2, and wherein the polypeptides of (f) bind and stimulate phosphorylation of VEGFR-3. In another aspect, the invention provides a polynucleotide comprising a nucleic acid sequence that encodes a polypeptide selected from the group consisting of (a) a polypeptide comprising amino acids 1-419 of SEQ ID NO: 2 or amino acids 32-419 of SEQ ID NO: 2; (b) a polypeptide comprising amino acids 103-227 of SEQ ID NO: 2, (c) a polypeptide comprising amino acids 131-211 of SEQ ID NO: 2; (d) a polypeptide comprising amino acids 32-227 of SEQ ID NO: 2; (e) a polypeptide comprising amino acids 103-419 of SEQ ID NO: 2; and (f) polypeptides according to (a)-(e), wherein a cysteine corresponding to position 156 of SEQ ID NO: 2 has been deleted or replaced with another amino acid, wherein the polypeptides of (a)-(e) bind and stimulate phosphorylation of VEGFR-3 and/or VEGFR-2, and wherein the polypeptides of (f) bind and stimulate phosphorylation of VEGFR-3.

Also provided is a polynucleotide comprising a nucleic acid sequence that encodes a polypeptide comprising an amino acid sequence at least 80%, 85%, 90%, 91%, 92%, 93%, 94%, 95%, 96%, 97%, 98%, 99%, 99.5%, or more identical to an amino acid sequence selected from the group consisting of (a) amino acids 1-354 of SEQ ID NO: 4 or amino acids 22-354 of SEQ ID NO: 4; (b) amino acids 93-201 of SEQ ID NO: 4, (c) amino acids 93-354 of SEQ ID NO: 4; (d) amino acids 22-201 of SEQ ID NO: 4; and (e) amino acids 92-201 of SEQ ID NO: 10, wherein the polypeptides of (a)-(5) bind and stimulate phosphorylation of VEGFR-3 and/or VEGFR-2. In another aspect, the invention provides a polynucleotide comprising a nucleic acid sequence comprising an amino acid sequence selected from the group consisting of (a) amino acids 1-354 of SEQ ID NO: 4 or amino acids 22-354 of SEQ ID NO: 4; (b) amino acids 93-201 of SEQ ID NO: 4, (c) amino acids 93-354 of SEQ ID NO: 4; (d) amino acids 22-201 of SEQ ID NO: 4; and (e) amino acids 92-201 of SEQ ID NO: 10.

In a related embodiment, the invention provides vectors comprising a polynucleotide of the invention. Such vectors are useful, e.g., for amplifying the polynucleotides in host cells to create useful quantities thereof, and for expressing polypeptides of the invention using recombinant techniques. In preferred embodiments, the vector is an expression vector wherein the polynucleotide of the invention is operatively linked to a polynucleotide comprising an expression control sequence. Autonomously replicating recombinant expression constructs such as plasmid and viral DNA vectors incorporating polynucleotides of the invention are specifically contemplated. Expression control DNA sequences include promoters, enhancers, and operators, and are generally selected based on the expression systems in which the expression construct is to be utilized. Preferred promoter and enhancer sequences are generally selected for the ability to increase gene expression, while operator sequences are generally selected for the ability to regulate gene expression. Expression vectors are useful for recombinant production of polypeptides of the invention. Expression constructs of the invention may also include sequences encoding one or more selectable markers that permit identification of host cells bearing the construct. Expression constructs may also include sequences that facilitate, and preferably promote, homologous recombination in a host cell. Preferred expression constructs of the invention also include sequences necessary for replication in a host cell.

Exemplary expression control sequences include promoter/enhancer sequences, (e.g., cytomegalovirus promoter/enhancer (Lehner et al., J. Clin. Microbiol., 29:2494-2502, 1991; Boshart et al., Cell, 41:521-530, 1985); Rous sarcoma virus promoter (Davis et al., Hum. Gene Ther., 4:151, 1993); Tie promoter (Korhonen et al., Blood, 86(5): 1828-1835, 1995); or simian virus 40 promoter for expression in the target mammalian cells, the promoter being operatively linked upstream (i.e. 5′) of the polypeptide coding sequence. In one embodiment, the promoter sequence comprises a tissue specific promoter such as a cardiac-specific or skeletal muscle-specific promoter. In another variation, the promoter is an epithelial-specific promoter or endothelial-specific promoter.

The polynucleotides of the invention may also optionally include a suitable polyadenylation sequence (e.g., the SV40 or human growth hormone gene polyadenylation sequence) operably linked downstream (i.e. 3′) of the polypeptide coding sequence.

The polynucleotides of the invention also optionally comprise a nucleotide sequence encoding a secretory signal peptide fused in frame with the polypeptide sequence. The secretory signal peptide directs secretion of the polypeptide of the invention by the cells that express the polynucleotide, and is cleaved by the cell from the secreted polypeptide. The signal peptide sequence can be that of a native VEGF-C or VEGF-D, or that of another secreted protein, or can be a completely synthetic signal sequence effective to direct secretion in cells of the mammalian subject.

The polynucleotide may further optionally comprise sequences whose only intended function is to facilitate large scale production of the vector, e.g., in bacteria, such as a bacterial origin of replication and a sequence encoding a selectable marker. However, in one embodiment, such extraneous sequences are at least partially cleaved off prior to administration to humans according to methods of the invention. One can manufacture and administer such polynucleotides for gene therapy using procedures that have been described in the literature for other transgenes. See, e.g., Isner et al., Circulation, 91: 2687-2692 (1995); and Isner et al., Human Gene Therapy, 7: 989-1011 (1996); incorporated herein by reference in their entirety.

In some embodiments, polynucleotides of the invention further comprise additional sequences to facilitate the gene therapy. In one embodiment, a “naked” transgene encoding a VEGF-C or VEGF-D growth factor product described herein (i.e. a transgene without a viral, liposomal, or other vector to facilitate transfection) is employed for gene therapy.

Vectors also are useful for “gene therapy” treatment regimens, wherein a polynucleotide that encodes a VEGF-C or VEGF-D growth factor product is introduced into a subject in need of treatment of hypertension, in a form that causes cells in the subject to express the VEGF-C or VEGF-D growth factor product of the invention in vivo. Gene therapy aspects that are described in commonly owned U.S. Patent Publication No. 2002/0151680 and WO 01/62942 both of which are incorporated herein by reference, also are applicable herein.

Any suitable vector may be used to introduce a polynucleotide that encodes a polypeptide of the invention encoding one of the polypeptides of the invention, into the host. Exemplary vectors that have been described in the literature include replication deficient retroviral vectors, including but not limited to lentivirus vectors (Kim et al., J. Virol., 72(1): 811-816,1998; Kingsman & Johnson, Scrip Magazine, October, 1998, pp. 43-46); adeno-associated viral (AAV) vectors (U.S. Pat. Nos. 5,474,9351; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89: 2581-2584, 1992; Stratford Perricadet et al., J. Clin. Invest., 90:626-630, 1992; and Rosenfeld et al., Cell, 68: 143-155, 1992); an adenoviral adeno-associated viral chimeric (U.S. Pat. No. 5,856,152) or a vaccinia viral or a herpesviral (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237, Liposomes comprising Sendai virus proteins); and combinations thereof. All of the foregoing documents are incorporated herein by reference in their entirety.

Other non-viral delivery mechanisms contemplated include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am. 276(6):102-6, 1997; Felgner, Hum Gene Ther. 7(15):1791-3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci. USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

The expression construct (or indeed the VEGF-C or VEGF-D growth factor products discussed above) may be entrapped in a liposome. Liposomes are vesicular structures characterized by a phospholipid bilayer membrane and an inner aqueous medium. Multi-lamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, Wu G, Wu C ed., New York: Marcel Dekker, pp. 87-104, 1991). The addition of DNA to cationic liposomes causes a topological transition from liposomes to optically birefringent liquid-crystalline condensed globules (Radler et al., Science, 275(5301):810-4, 1997). These DNA-lipid complexes are potential non-viral vectors for use in gene therapy and delivery.

Liposome-mediated nucleic acid delivery and expression of foreign DNA in vitro has been successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads.

In embodiments employing a viral vector, preferred polynucleotides still include a suitable promoter and polyadenylation sequence as described above. Moreover, it will be readily apparent that, in these embodiments, the polynucleotide further includes vector polynucleotide sequences (e.g., adenoviral polynucleotide sequences) operably connected to the sequence encoding a polypeptide of the invention.

In another embodiment, the invention provides host cells, including prokaryotic and eukaryotic cells, that are transformed or transfected (stably or transiently) with polynucleotides of the invention or vectors of the invention. Polynucleotides of the invention may be introduced into the host cell as part of a circular plasmid, or as linear DNA comprising an isolated protein coding region or a viral vector. Methods for introducing DNA into the host cell, which are well known and routinely practiced in the art, include transformation, transfection, electroporation, nuclear injection, or fusion with carriers such as liposomes, micelles, ghost cells, and protoplasts. As stated above, such host cells are useful for amplifying the polynucleotides and also for expressing the polypeptides of the invention encoded by the polynucleotide. The host cell may be isolated and/or purified. The host cell also may be a cell transformed in vivo to cause transient or permanent expression of the polypeptide in vivo. The host cell may also be an isolated cell transformed ex vivo and introduced post-transformation, e.g., to produce the polypeptide in vivo for therapeutic purposes. The definition of “host cell” explicitly excludes a transgenic human being.

For expression of polypeptides of the invention, any host cell is acceptable, including but not limited to bacterial, yeast, plant, invertebrate (e.g., insect), vertebrate, and mammalian host cells. For developing therapeutic preparations, expression in mammalian cell lines, especially human cell lines, is preferred. Use of mammalian host cells is expected to provide for such post-translational modifications (e.g., glycosylation, truncation, lipidation, and phosphorylation) as may be desirable to confer optimal biological activity on recombinant expression products of the invention. Glycosylated and non-glycosylated forms of polypeptides are embraced by the present invention. Similarly, the invention further embraces polypeptides described above that have been covalently modified to include one or more water soluble polymer attachments such as polyethylene glycol, polyoxyethylene glycol, or polypropylene glycol.

In another embodiment, the invention provides epithelial or endothelial cells or progenitor cells transformed or transfected ex vivo with the gene(s) encoding a VEGF-C or VEGF-D growth factor product, and the transfected cells as administered to the mammalian subject.

Similarly, the invention provides for the use of polypeptides or polynucleotides or host cells of the invention in the manufacture of a medicament for the treatment of disorders described herein, including but not limited to disorders associated with hypertension.

In a related embodiment, the invention provides a kit comprising a polynucleotide, polypeptide, or composition of the invention packaged in a container, such as a vial or bottle, and further comprising a label attached to or packaged with the container, the label describing the contents of the container and providing indications and/or instructions regarding use of the contents of the container to treat one or more disease states as described herein.

### IV. THERAPEUTIC USES OF THE VEGF-C AND VEGF-D GROWTH FACTOR PRODUCTS

In yet another embodiment, the invention provides numerous in vitro and in vivo methods of using the VEGF-C and VEGF-D growth factor products of the invention. Generally speaking, the VEGF-C or VEGF-D growth factor products of the invention are useful for reducing hypertension in a subject.

Thus, in one variation, the invention provides a method of prophylaxis or therapy for a method of treating a mammalian subject suffering from hypertension, comprising: administering to said subject a composition comprising at least one therapeutic agent selected from the group consisting of a polynucleotide comprising a nucleotide sequence that encodes a vascular endothelial growth factor C (VEGF-C) growth factor product or a vascular endothelial growth factor D (VEGF-D) growth factor product, wherein said composition is administered in an amount effective to reduce systolic or diastolic blood pressure in said subject.

Hypertension is classified as follows:

| Blood pressure | |
|---|---|
| (mmHg) | Status of Hypertension |
| <120/80 | Normal in a healthy individual |
| 120-139/80-89 | Prehypertension |
| >140/90 | Hypertension |

A desirable level of systolic or diastolic blood pressure reduction can be determined by those skilled in the art. For example, in some embodiments, a reduction in blood pressure includes the normalization of the subject's blood pressure to a systolic blood pressure of below 120 mm Hg, a diastolic blood pressure of about 80 mm Hg or a combination of a systolic blood pressure of below 120 mm Hg and a diastolic blood pressure of 80 mmHg. For individuals with diabetes or chronic kidney disease, the recommended blood pressure for these individuals is <130/80 mmHg. In some embodiments, any reduction in systolic or diastolic blood pressure is considered a beneficial result.

In some embodiments, the methods may involve establishing an initial or baseline blood pressure (such as a systolic blood pressure, a diastolic blood pressure, a mean arterial blood pressure or a combination of a systolic blood pressure and a diastolic blood pressure) for a subject. Methods for determining the blood pressure of a subject are well known in the art. For example, the systolic blood pressure and/or diastolic blood pressure of a subject can be determined using a sphygmomanometer (in mm of Hg) by a medical professional, such as a nurse or physician. Aneroid or electronic devices can also be used to determine the blood pressure of a subject and these devices and their use are also well known to those skilled in the art. Additionally, a 24-hour ambulatory blood pressure monitoring (hereinafter “ABPM”) device can be used to measure systolic blood pressure, diastolic blood pressure and heart rate. ABPM assesses systolic blood pressure, diastolic blood pressure and heart rate in predefined intervals (normally, the intervals are established at every 15 or 20 minutes, but any interval can be programmed) over a 24-hour period. The time at which the blood pressure of the subject is determined is not critical for establishing the initial or baseline blood pressure reading. Once the initial or baseline blood pressure reading has been determined, a further determination is made by those skilled in the art as to whether or not the subject is suffering from (a) pre-hypertension; or (b) hypertension.

Dose-response studies permit accurate determination of a proper quantity of the VEGF-C and/or VEGF-D growth factor product to employ. Effective quantities can be estimated from measurements of the binding affinity of a polypeptide for a target receptor, of the quantity of receptor present on target cells, of the expected dilution volume (e.g., patient weight and blood volume for in vivo embodiments), and of polypeptide clearance rates. Existing literature regarding dosing of known VEGF-C and VEGF-D also provides guidance for dosing of VEGF-C or VEGF-D growth factor products of the invention.

Generally speaking, embodiments described herein in the context of administering polypeptides can also be practiced by administering polynucleotides that encode the polypeptides. Polynucleotide therapy (e.g., using gene therapy vectors) may result in sustained production of a construct in vivo, reducing or eliminating the need for repeated dosing of polypeptides.

Polynucleotides or polypeptides of the invention can be administered purely as a prophylactic treatment to prevent hypertension in subjects at risk for developing hypertension, or as a therapeutic treatment to subjects afflicted with hypertension, for the purpose of reducing systolic or diastolic blood pressure in said subjects.

In another embodiment, the invention provides a method of treating hypertension in a subject in need thereof comprising identifying a subject as being resistant to treatment with a standard of care anti-hypertensive agent and administering a VEGF-C and or VEGF-D growth factor product to the subject. A subject is considered resistant to treatment when the blood pressure of a subject remains elevated above treatment goals despite administration of a three drug treatment regimen that includes a diuretic (Nuesch et al., British Medical Journal, 323:12-146, 2001). Because some cases of blood pressure are difficult to treat, and may require a combination of multiple drugs before control is established, high blood pressure cannot be called “resistant” until this three-drug combination therapy has failed.

In one variation, the methods optionally further comprise administering a standard of care regimen for the treatment of hypertension. In the context of methods of the invention, “standard of care” refers to a treatment that is generally accepted by clinicians for a certain type of patient diagnosed with a type of illness. For hypertension, for example, an aspect of the invention is to improve standard of care therapy with co-therapy with VEGF-C and/or VEGF-D growth factor products described herein. Exemplary standard of care therapeutics for hypertension include, but are not limited to, Coenzyme Q10; renin inhibitors (e.g., aliskiren); angiotensin-convertin enzyme (ACE) inhibitors (e.g., captopril, enalapril maleate, ramipril, meoxipril, quinapril hydrochloride, lisinopril, benazepril hydrochloride, trandolapril and fosinopril sodium); angiotensin II receptor (AR) blockers (e.g., losartan potassium, candesartan, irbesartan, eprosartan, olmesartan, valsartan, and telmisartan), alpha blockers (e.g., doxazosin, prazosin, and terazosin); diuretics (e.g., hydrochlorothiozide, bendroflumethiazide, spironolactone, amiloride hydrochloride, triamterene, furosemide, torsemide, bumetanide, and ethacrynic acid); beta blockers (e.g., acebutolol, bisoprolal, carteolol, carvedilo, celiprolol, labetalol, mepindolol, metoprolol, oxprenolol, pindolol, atenolol, celiprolol, nadolol, nebivolol, sotalol, timolol, betaxolol, propranolol, and carvedilol); and calcium channel blockers (e.g., amlodipine besylate, felodipine, isradipine, nicardipine, nifedipine, nimodipine, nisoldipine, nitrendipine, lacidipine, lercanidipine, verapamil hydrochloride, gallopamil, and diltiazem hydrochloride).

In another embodiment, the invention provides a method of treating hypertension in a subject comprising administering a VEGF-C or VEGF-D growth factor product to a subject in need thereof, wherein the subject has been identified as having secondary hypertension. In some embodiments, the identifying comprises determining whether the subject has a disorder or condition selected form the group consisting of pregnancy, cancer, polycystic kidney disease, chronic glomerulonephritis, disease of the renal arteries, aldosteronism, Cushing's syndrome and pheochromocytoma.

Also contemplated are methods of treating a subject with hypertension that is hypo-responsive to a standard of care regimen for the treatment of hypertension comprising administering a VEGF-C or VEGF-D growth factor product to the subject.

In a preferred embodiment, the mammalian subject is a human subject. Practice of methods of the invention in other mammalian subjects, especially mammals that are conventionally used as models for demonstrating therapeutic efficacy in humans (e.g., primate, porcine, canine, or rabbit animals), is also contemplated. In one aspect, the subject is a person suffering from hypertension.

In some embodiments, the methods optionally further comprise a step, prior to the administering step, of selecting as the subject for treatment or prophylaxis a human with one or more conditions selected from the group consisting of pregnancy, cancer, polycystic kidney disease, chronic glomerulonephritis, disease of the renal arteries, aldosteronism, Cushing's syndrome and pheochromocytoma

### V. COMBINATION THERAPY

Combination therapy embodiments of the invention include products and methods. Exemplary combination products include two or more agents formulated as a single composition or packaged together in separate compositions, e.g., as a unit dose package or kit. Exemplary combination methods include prescribing for administration, or administration of two or more agents simultaneously or in tandem.

A combination of a VEGF-C or VEGF-D growth factor product with one or more additional therapeutics/second agents in methods of the invention may reduce the amount of either agent needed as a therapeutically effective dosage, and thereby reduce any negative side effects the agents may induce in vivo. Combination therapy preferably results in improved efficiency compared to either agent alone.

In one embodiment, methods described herein optionally further comprise administering a standard of care therapeutic to the subject. In some embodiments, the standard of care therapeutic and the VEGF-C and/or VEGF-D growth factor product are co-administered in a single composition. In other embodiments, the standard of care therapeutic is administered as a separate composition from the VEGF-C and/or VEGF-D growth factor product. Exemplary standard of care therapeutics for hypertension include, but are not limited to, Coenzyme Q10; renin inhibitors (e.g., aliskiren); angiotensin-convertin enzyme (ACE) inhibitors (e.g., captopril, enalapril maleate, ramipril, meoxipril, quinapril hydrochloride, lisinopril, benazepril hydrochloride, trandolapril and fosinopril sodium); angiotensin II receptor (AR) blockers (e.g., losartan potassium, candesartan, irbesartan, eprosartan, olmesartan, valsartan, and telmisartan), alpha blockers (e.g., doxazosin, prazosin, and terazosin); diuretics (e.g., hydrochlorothiozide, bendroflumethiazide, spironolactone, amiloride hydrochloride, triamterene, furosemide, torsemide, bumetanide, and ethacrynic acid); beta blockers (e.g., acebutolol, bisoprolal, carteolol, carvedilo, celiprolol, labetalol, mepindolol, metoprolol, oxprenolol, pindolol, atenolol, celiprolol, nadolol, nebivolol, sotalol, timolol, betaxolol, propranolol, and carvedilol); and calcium channel blockers (e.g., amlodipine besylate, felodipine, isradipine, nicardipine, nifedipine, nimodipine, nisoldipine, nitrendipine, lacidipine, lercanidipine, verapamil hydrochloride, gallopamil, and diltiazem hydrochloride).

In another embodiment, methods described herein optionally further comprise administering a VEGF growth factor product to the subject. In some embodiments, a VEGF growth factor product comprises a VEGF-A polypeptide (or a VEGF-A polynucleotide that encodes a VEGF-A polypeptide) that binds and stimulates phosphorylation of VEGFR-1 and/or VEGFR-2. VEGF-A (SEQ ID NOs: 12 (polynucleotide sequence) and 13 (amino acid sequence)) is a secreted, disulfide-linked homodimeric glycoprotein composed of 23 kD subunits. Five human VEGF-A isoforms of 121 (SEQ ID NO: 14), 145 (SEQ ID NO: 15), 165 (SEQ ID NO: 16), 189 (SEQ ID NO: 17) or 206 (SEQ ID NO: 18) amino acids in length (VEGF₁₂₁₋₂₀₆), encoded by distinct mRNA splice variants, have been described, all of which are capable of stimulating mitogenesis in endothelial cells. However, each isoform differs in biological activity, receptor specificity, and affinity for cell surface- and extracellular matrix-associated heparan-sulfate proteoglycans, which behave as low affinity receptors for VEGF-A. VEGF₁₂₁ does not bind to either heparin or heparan-sulfate; VEGF₁₄₅ and VEGF₁₆₅ (GenBank Acc. No. M32977) are both capable of binding to heparin; and VEGF₁₈₉ and VEGF₂₀₆ show the strongest affinity for heparin and heparan-sulfates. VEGF₁₂₁, VEGF₁₄₅, and VEGF₁₆₅ are secreted in a soluble form, although most of VEGF₁₆₅ is confined to cell surface and extracellular matrix proteoglycans, whereas VEGF₁₈₉ and VEGF₂₀₆ remain associated with extracellular matrix. Both VEGF₁₈₉ and VEGF₂₀₆ can be released by treatment with heparin or heparinase, indicating that these isoforms are bound to extracellular matrix via proteoglycans. Cell-bound VEGF₁₈₉ can also be cleaved by proteases such as plasmin, resulting in release of an active soluble VEGF₁₁₀. Most tissues that express VEGF are observed to express several VEGF isoforms simultaneously, although VEGF₁₂₁ and VEGF₁₆₅ are the predominant forms, whereas VEGF₂₀₆ is rarely detected (Ferrara, J Mol Med 77:527-543, 1999). VEGF₁₄₅ differs in that it is primarily expressed in cells derived from reproductive organs (Neufeld et al., FASEB J 13:9-22, 1999).

As noted above, the human VEGF-A gene is expressed as numerous isoforms, including VEGF₁₄₅, VEGF₁₆₅, VEGF₁₈₉, and VEGF₂₀₆. A human VEGF₂₀₆ sequence obtained from the Swiss Prot database (accession no. P15692) is set forth below and in SEQ ID NO: 18:

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksry kswsvyvgar cclmpwslpg |
| 181 | phpcgpcser rkhlfvqdpq tckcsckntd srckarqlel nertcrcdkp rr |

Amino acids 1-26 of this sequence represent the signal peptide and mature VEGF₂₀₆ comprises amino acids 27-232. Referring to the same sequence, the signal peptide and amino acids 142-226 are absent in mature isoform VEGF₁₂₁ (SEQ ID NO: 14). The signal peptide and amino acids 166-226 are absent in mature isoform VEGF₁₄₅ (SEQ ID NO: 15). The signal peptide and amino acids 142-182 are absent in mature isoform VEGF₁₆₅ (SEQ ID NOs: 16). The signal peptide and amino acids 166-182 are absent in mature isofrom VEGF₁₈₉ (SEQ ID NO.: 17).

VEGF₁₀₉, which comprises only the VEGF homology domain (i.e., the minimal receptor binding domain), has been tested for angiogenic activity in a chick CAM assay but results indicated that it was less angiogenic than VEGF₁₆₅ (Jeltsch et al., J. Biol. Chem., 281, 12187-95, 2006).

In some embodiments, VEGF-A polypeptides for use in a method described herein comprise an amino acid sequence at least 80%, 85%, 90%, 91%, 92%, 93%, 94%, 95%, 96%, 97%, 98%, 99%, 99.5%, or more identical to a polypeptide comprising an amino acid sequence selected from the group consisting of SEQ ID NOs: 13, 14, 15, 16, 17 and 18, wherein the polypeptide binds and stimulates at least one of VEGFR-1 and VEGFR-2. In some embodiments, the polypeptide binds and stimulates both VEGFR-1 and VEGFR-2.

Combination therapy with one or more of the additional agents described herein may be achieved by administering to a subject a single composition or pharmacological formulation that includes the VEGF-C or VEGF-D growth factor product and the one or more additional agents, or by administering to the subject two (or more) distinct compositions or formulations, at the same time, wherein one composition includes an VEGF-C or VEGF-D growth factor product and the other includes a second agent.

Alternatively, the combination therapy employing a VEGF-C or VEGF-D growth factor product described herein may precede or follow the second agent treatment by intervals ranging from minutes to weeks. In embodiments where the second agent and the VEGF-C or VEGF-D growth factor product are administered separately, one would generally ensure that a significant period of time did not expire between the times of each delivery, such that the agent and the VEGF-C or VEGF-D growth factor product would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Repeated treatments with one or both agents is specifically contemplated.

### VI. COMPOSITIONS AND FORMULATIONS

Compositions for use in accordance with the present invention may be formulated in a conventional manner using one or more physiologically acceptable carriers comprising excipients and auxiliaries which facilitate processing of a therapeutic composition into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

Polypeptides and/or polynucleotides of the invention may be administered in any suitable manner using an appropriate pharmaceutically acceptable vehicle, e.g., a pharmaceutically acceptable diluent, adjuvant, excipient or carrier. The composition to be administered according to methods of the invention preferably comprises (in addition to the polypeptide, polynucleotide or vector) a pharmaceutically acceptable carrier solution such as water, saline, phosphate buffered saline, glucose, or other carriers conventionally used to deliver therapeutics orally or systemically.

The “administering” may be performed using any medically-accepted means for introducing a therapeutic directly or indirectly into the vasculature of a mammalian subject, including but not limited to injections (e.g., intravenous, intramuscular, subcutaneous, or catheter); oral ingestion; intranasal or topical administration; and the like. The therapeutic composition may be delivered to the patient at multiple sites.

When a therapeutically effective amount of a composition of the present invention is administered by e.g., intradermal, cutaneous or subcutaneous injection, the composition is preferably in the form of a pyrogen-free, parenterally acceptable aqueous solution. The preparation of such parenterally acceptable protein or polynucleotide solutions, having due regard to pH, isotonicity, stability, and the like, is within the skill in the art.

A preferred composition should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The composition of the present invention may also contain stabilizers, preservatives, buffers, antioxidants, or other additives known to those of skill in the art. The agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compounds with pharmaceutically acceptable carriers well known in the art. Such carriers enable the compounds of the invention to be formulated as tablets, pills, dragees, powders, capsules, liquids, solutions, gels, syrups, slurries, suspensions and the like, for oral ingestion by a patient to be treated.

The therapeutic composition may be delivered to the patient via one or more routes of administration. The multiple administrations may be rendered simultaneously or may be administered over a period of several hours or days. Additional therapy may be administered on a period basis, for example, daily, weekly or monthly.

The amounts of VEGF-C and/or VEGF-D growth factor product in a given dosage will vary according to the size of the individual to whom the therapy is being administered as well as the characteristics of the disorder being treated. Dose is adjusted to achieve therapeutic or prophylactic benefit while minimizing side effects. In exemplary treatments, it may be necessary to administer between about 10 μg/day to about 250 mg/day. In some embodiments, it may be necessary to administer about 10 μg/day, 25 μg/day, 50 μg/day, 75 μg/day, 100 μg/day, 125 μg/day, 150 μg/day, 175 μg/day, 200 μg/day, 225 μg/day, 250 μg/day, 275 μg/day, 300 μg/day, 325 μg/day, 350 μg/day, 375 μg/day, 400 μg/day, 425 μg/day, 450 μg/day, 475 μg/day, 500 μg/day, 750 μg/day, 1 mg/day, 5 mg/day, 10 mg·day, 25 mg/day, 30 mg/day, 40 mg/day, 45 mg/day, 50 mg/day, 60 mg/day, 70 mg/day, 80 mg/day, 90 mg/day, 100 mg/day, 150 mg/day, 200 mg/day or about 250 mg/day. In some embodiments, the maximum dosage is 200 mg/day. These concentrations may be administered as a single dosage form or as multiple doses.

The compositions also may comprise suitable solid or gel phase carriers or excipients.

The compositions may include a matrix capable of delivering the protein-containing or other active ingredient-containing composition to the site of tissue damage. Such matrices may be formed of materials presently in use for other implanted medical applications. The choice of matrix material is based on biocompatibility, biodegradability, mechanical properties, cosmetic appearance and interface properties.

The composition may further contain other agents which either enhance the activity of the protein or other active ingredient or complement its activity or use in treatment. Such additional factors and/or agents may be included in the pharmaceutical composition to produce a synergistic effect with protein or other active ingredient of the invention, or to minimize side effects. VEGF-C and -D proteins form dimers and as a result, pharmaceutical compositions of the invention may comprise a protein of the invention in such multimeric or in complexed forms.

Techniques for formulation and administration of the therapeutic compositions of the instant application may be found in “Remington's Pharmaceutical Sciences,” Mack Publishing Co., Easton, Pa., latest edition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

### VII. KITS

Kits which comprise compounds or compositions of the invention packaged in a manner which facilitates their use to practice methods of the invention are also contemplated. In a simplest embodiment, such a kit includes a VEGF-C and/or VEGF-D growth factor product or composition described herein as useful for practice of the invention (e.g., polynucleotides or polypeptides of the invention), packaged in a container such as a sealed bottle or vessel, with a label affixed to the container or included in the package that describes use of the compound or composition to practice the method of the invention. Preferably, the compound or composition is packaged in a unit dosage form. In another embodiment, a kit of the invention includes a composition of a polynucleotide and/or polypeptide packaged together with a physical device useful for implementing methods of the invention, such as a stent, a catheter, a polymer film, or the like. In another embodiment, a kit of the invention includes compositions of a polynucleotide and/or polypeptide of the invention packaged together with a hydrogel polymer, or microparticle polymers, or other carriers described herein as useful for delivery of the polynucleotides or polypeptides to the patient.

### VIII. EXAMPLES

### Example 1

### VEGF-C Significantly Reduced Blood Pressure in Mice

Methods: 8 weeks old male ICR mice were anesthetized with 30 mg/kg phenobarbital, which does not have direct effects on blood pressure. The mice were placed in a +35° C. incubator to stimulate peripheral blood flow, and blood pressure measurement cuffs were placed around the root of the tail. Baseline blood pressure levels were determined from three consecutive measurements yielding a consistent result (within 10 mmHg). 400 ng, 1μg, 3 μg or 10 μg of recombinant human (rh) VEGF-CΔNΔC was administered into the tail vein of mice at a volume of 200 μl, and blood pressure was measured dynamically at 1-minute intervals.

Immunocytochemistry: Porcine Aortic Endothelial (PAE) cells overexpressing either VEGFR-2 (PAE-KDR) (Waltenberger et al., J. Biol. Chem., 269:26988-26995, 1994), or VEGFR-3 (PAE-FLT4) (Pajusola et al., Oncogene, 9:3545-3555, 1994) were used for the stimulation experiment. Cells were starved for 2 hours in F12 growing medium with FCS and stimulated for 20 minutes at +37° C. with human VEGF-A₁₆₅ (50 ng/mL) or human VEGF-C_{ΔNΔC} (100 ng/mL), while BSA was used as a negative control. After stimulation cells were fixed for 10 minutes in 4% paraformaldehyde-PBS, permeabilized for 5 minutes with 0.1% Triton X-100 in PBS, blocked for 10 minutes in 1% BSA-PBS, and incubated with primary antibodies against human VEGFR-3, human VEGFR-2 and phosphor-eNOS in 1% BSA-PBS for 1 hour at room temperature. The cells were then incubated with secondary antibodies for 20 minutes at room temperature. Images were captured using a laser scanning confocal microscope (Zeiss LSM 510 Meta).

Western blotting: After overnight incubation with 0.5% FBS media, human dermal microvascular endothelial cells (HDMEC) were stimulated for 15 minutes with VEGF-A, VEGF-C or mix of VEGF-A and VEGF-C. Cells were lysed in lysis buffer (1% Nonidet P-40, 20 mM Tris-HCl (pH 7.5), 150 mM NaCl, 5 mM EDTA, 2 mM NA₃VO₄, 100 μM PMSF and 10 μg/mL each of aprotinin and leupeptin), and insoluble materials were removed by centrifugation (15,000g for 5 minutes). Subsequently, lysates were blotted with phospho-eNOS and eNOS antibodies or phospho-AKT and AKT antibodies. All antibodies were from BD Biosciences.

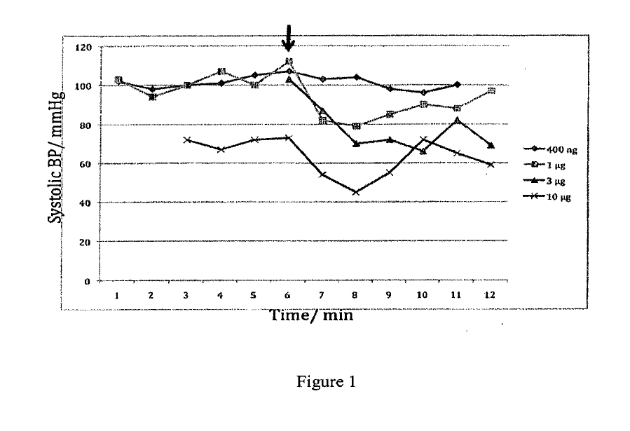

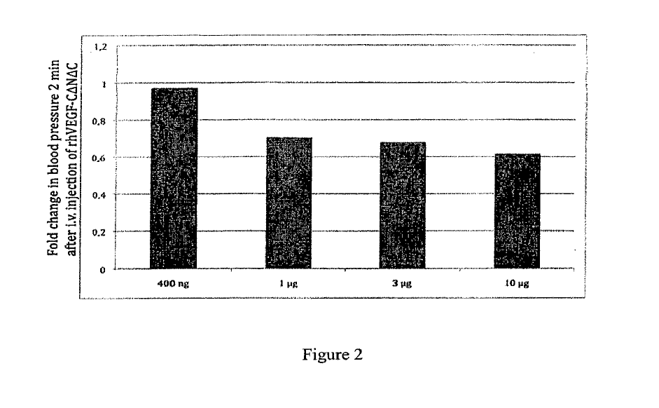

Results: It was determined that injection of VEGF-CΔNΔC decreased systolic blood pressure by approximately 20 mmHg within 1 minute from the injection at doses of 1 μg and higher (see FIG. 1). Maximal decrease in blood pressure was observed at 2 minutes (see FIG. 2). The decrease in blood pressure appeared to be more dramatic at the 10 μs dose, when compared to 1 μg and 3 μg, as baseline levels in this particular mouse were lower. The blood pressure returned to baseline levels in approximately 4-5 minutes, suggesting that VEGF-C is rapidly inactivated in the blood, and/or that a compensatory sympathetic response involving catecholamine release is initiated. No edema or shortness of breath was observed in mice injected with the growth factors.

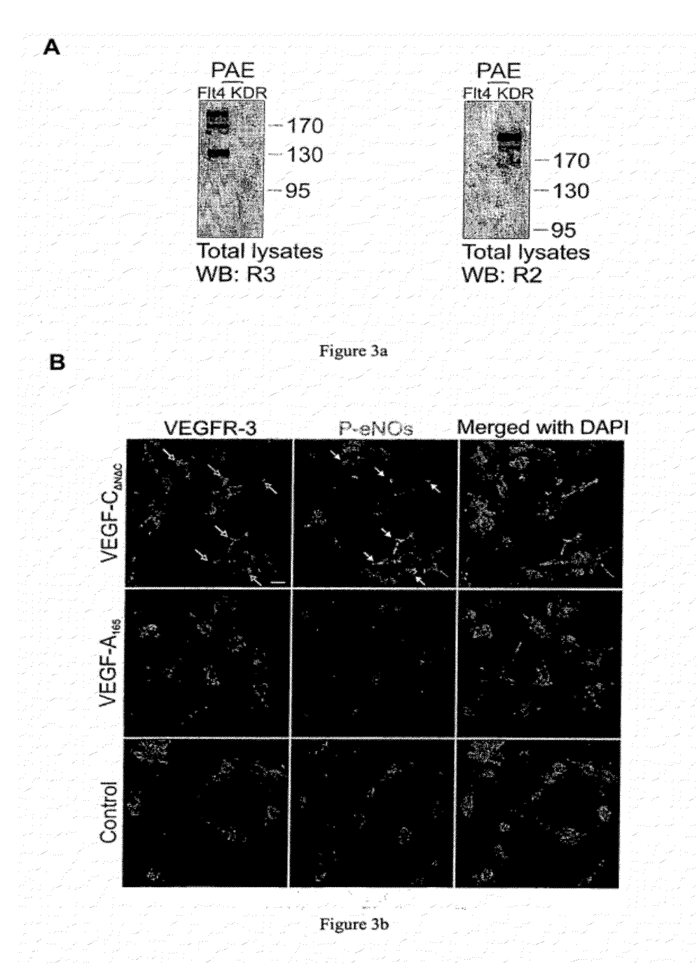

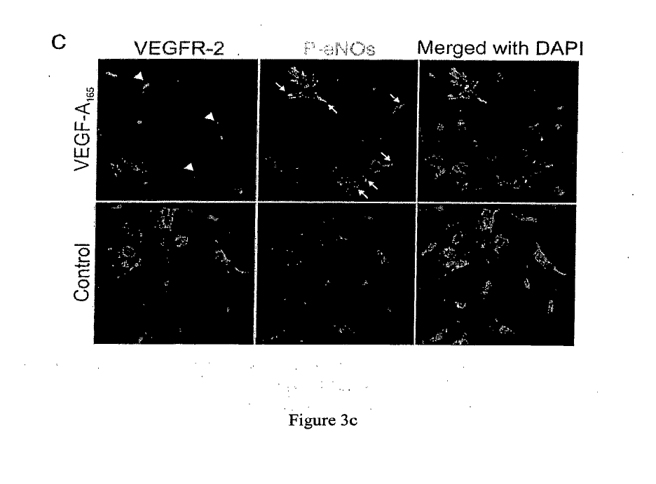

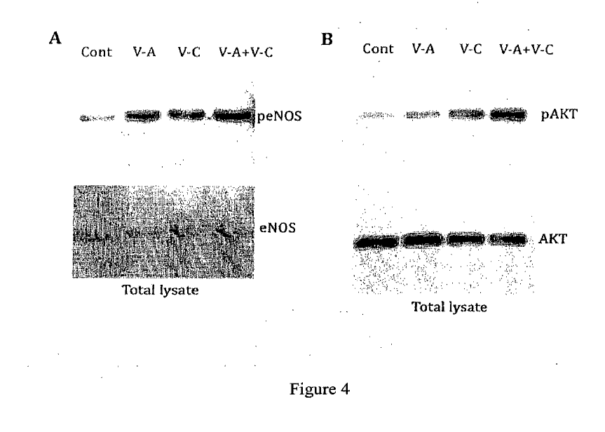

Cell culture experiments revealed that stimulation of cells that express only VEGFR-3 with VEGF-C promoted the activation (phosphorylation) of eNOS (see FIGS. 3A and 3B). Activation of VEGFR-2 with VEGF in cells that express exclusively this receptor also led to phosphorylation of eNOS. See FIG. 3C. VEGF-C also promoted eNOS phosphorylation in HDMEC's, which are primary human endothelial cells (see FIG. 4). Interestingly, VEGF-C appeared to synergize with VEGF, as combined administration of the two factors led to highly pronounced eNOS activation.

Discussion: Results demonstrated that VEGF-C can decrease blood pressure in adult mice. Based on these findings, VEGF-C could also be used in the human setting to treat hypertension, and possibly offer a treatment modality for patients that respond poorly or are refractory to currently available anti-hypertensive pharmaceuticals (Ram, Current Hypertension Reports, 8:398-402, 2006). The decrease observed in blood pressure resulting from short-term stimulation with VEGF-CΔNΔC is probably due to increased release of nitric oxide as a result of VEGFR-2 activation. However, long-term stimulation with wild-type and VEGFR-3 specific forms of VEGF-C will also stimulate lymphangiogenesis (Alitalo et al., Nature, 2005, 438:946-53), which may increase the salt buffering capacity of the lymphatics, which may lead to long-term reduction in blood pressure. Importantly, the administration of even high doses of VEGF-C did not result in edema or difficulties in breathing, indicating that VEGF-C is potentially safe to use also in human patients. The apparent synergistic effects of VEGF-C and VEGF-A co-administration provide an indication that such co-therapy may be effective in vivo. Co-therapy of VEGF-C or -D with a VEGFR-2 ligand such as VEGF-A is contemplated as a variation of the invention. Combination therapy (e.g., co-administration or co-formulation with other anti-ischemia agents, such as glycerylnitrate) is also contemplated as part of the invention.

The acute onset of myocardial ischemia is typically treated with nitric oxide donors, such as glycerylnitrate (to relax coronary arteries) which improves perfusion in the ischemic myocardium. Administering recombinant VEGF-CΔNΔC could be used to increase the endogenous synthesis of nitric oxide to promote perfusion in such a setting, or in other tissues (e.g., brain) that are foci of localized acute ischemia. Treatment of acute ischemia with VEGF-C or VEGF-D products is another aspect of the invention.

### Example 2

### Other Forms of VEGF-C Reduce Blood Pressure in Mice

The method of Example 1 is repeated using recombinant human VEGF-CΔC₁₅₆, VEGF-CΔN and VEGF-CΔC. The administration of any of these growth factor products is expected to reduce blood pressure in mice in a manner similar to that of VEGF-CΔNΔC.

### Example 3

### VEGF-D Reduces Blood Pressure in Mice

The method of Example 1 is repeated using recombinant human VEGF-DΔNΔC, VEGF-DΔN and VEGF-DΔC. The administration of any of these growth factor products is expected to reduce blood pressure in mice in a manner similar to that of VEGF-CΔNΔC.

1. A method of treating a mammalian subject suffering from hypertension, comprising:
administering to said subject a composition comprising at least one therapeutic agent selected from the group consisting of a VEGF-C growth factor product and a VEGF-D growth factor product, wherein said composition is administered in an amount effective to reduce systolic or diastolic blood pressure in said subject.

2-4. (canceled)

5. The method according to claim 1, wherein said mammalian subject is human.

6. The method according to claim 5, wherein the at least one therapeutic agent comprises a VEGF-C growth factor product that comprises a VEGF-C polypeptide.

7. The method according to claim 6, wherein the VEGF-C polypeptide comprises a member selected from the group consisting of a full length VEGF-C polypeptide, a VEGF-CΔNΔC polypeptide, a VEGF-CΔN polypeptide, and a VEGF-CΔC polypeptide.

8. (canceled)

9. The method according to claim 6, wherein the VEGF-C polypeptide is selected from the group consisting of a polypeptide selected from the group consisting of:
(a) a polypeptide comprising amino acids 1-419 of SEQ ID NO: 2 or a polypeptide comprising amino acids 32-419 of SEQ ID NO: 2;
(b) a polypeptide comprising amino acids 103-227 of SEQ ID NO: 2;
(c) N-terminal deletion fragments of (a) or (b) that bind and stimulate phosphorylation of VEGFR-2 and/or VEGFR-3;
(d) C-terminal deletion fragments of (a) or (b) or (c) that bind and stimulate phosphorylation of VEGFR-2 and/or VEGFR-3 ;
(e) polypeptides that comprise an amino acid sequence at least 90% identical to (a) or (b) or (c) or (d) that bind and stimulate phosphorylation of VEGFR-2 and/or VEGFR-3; and
(f) polypeptides according to (a), (b), (c), (d), or (e), wherein the cysteine corresponding to position 156 of SEQ ED NO: 2 has been deleted or replaced with another amino acid and wherein the polypeptides bind and stimulate phosphorylation of VEGFR-3.

10. (canceled)

11. The method according to claim 5, wherein the at least one therapeutic agent comprises a VEGF-D growth factor product that comprises a VEGF-D polypeptide.

12. The method according to claim 11, wherein the VEGF-D polypeptide is selected from the group consisting of a full length VEGF-D polypeptide, a VEGF-DΔNΔC polypeptide, a VEGF-DΔN polypeptide, and a VEGF-DΔC polypeptide.

13. (canceled)

14. The method according to claim 11, wherein the VEGF-D polypeptide is selected from the group consisting of:
(a) a polypeptide comprising amino acids 1-354 of SEQ ID NO: 4 or a polypeptide comprising amino acids 22-354 of SEQ ID NO: 4;
(b) a polypeptide comprising amino acids 93-201 of SEQ ID NO: 4;
(c) N-terminal deletion fragments of (a) or (b) that bind and stimulate phosphorylation of VEGFR-2 and/or VEGFR-3;
(d) C-terminal deletion fragments of (a) or (b) or (c) that bind and stimulate phosphorylation of VEGFR-2 and/or VEGFR-3; and
(e) polypeptides that comprise an amino acid sequence at least 90% identical to (a) or (b) or (c) or (d) that bind and stimulate phosphorylation of VEGFR-2 and/or VEGFR-3.

15. The method according to claim 5, wherein the at least one therapeutic agent comprises a VEGF-C growth factor product that is a polynucleotide comprising a nucleotide sequence that encodes a VEGF-C polypeptide.

16. The method according to claim 5, wherein the at least one therapeutic agent comprises a VEGF-D growth factor product that is a polynucleotide comprising a nucleotide sequence that encodes a VEGF-D polypeptide.

17. The method according to claim 15, wherein the composition comprises a vector comprising the polynucleotide operatively connected to at least one expression control sequence.

18. The method according to claim 17, wherein the vector comprises a replication-deficient viral vector.

19. The method according to claim 18, wherein the vector comprises a member selected from the group consisting of adenoviral vectors, adeno-associated virus vectors, lentivirus vectors, herpes virus vectors, and vaccinia virus vectors.

20. The method according to claim 5, wherein the subject suffers from hypertension and the method further comprises administering at least one standard of care anti-hypertensive agent to the subject.

21. The method according to claim 20, wherein the standard of care anti-hypertensive agent is selected from the group consisting of Coenzyme Q10, renin inhibitors, angiotensin-convertin enzyme (ACE) inhibitors, angiotensin II receptor blockers (ARBs), alpha blockers, diuretics, beta blockers and calcium channel blockers.

22-36. (canceled)

37. The method according to claim 5, wherein the method further comprises administering a VEGF growth factor product to the subject.

38. The method according to claim 5, wherein the composition further comprises a pharmaceutically-acceptable carrier, excipient or diluent.

39-44. (canceled)

45. A method of treating hypertension in a subject, the method comprising
(a) identifying a subject as being resistant to treatment with a standard of care anti-hypertensive agent; and
(b) administering to said subject a composition comprising at least one therapeutic agent selected from the group consisting of a VEGF-C growth factor product and a VEGF-D growth factor product, wherein said composition is administered in an amount effective to reduce systolic or diastolic blood pressure in said subject.

46. The method according to claim 45, wherein the identifying comprises measuring the blood pressure of a subject before and after combination therapy with at least three anti-hypertensive agents, wherein at least one of the anti-hypertensive agents is a diuretic, and
wherein the continued elevation of blood pressure of the subject after the combination therapy identifies the subject as being resistant to treatment with a standard of care anti-hypertensive agent.

47-52. (canceled)

53. A composition that comprises a VEGF-D therapeutic or a VEGF-C therapeutic in admixture with a standard of care anti-hypertensive therapeutic.

54-59. (canceled)
